(19) United States
(12) Patent Application Publication
(10) Pub. No.: US 2004/0005549 A1
(43) Pub. Date: Jan. 8, 2004

(54) Hepatitis C virus constructs characterized by high efficiency replication

(75) Inventors: Vadim Bichko, Belmont, MA (US); Victor Shifrin, Newton, MA (US); Svetlana Bergelson, Newton, MA (US)

Correspondence Address: DARBY & DARBY P.C., P. O. BOX 5257, NEW YORK, NY, 10150-5257, US

(73) Assignee: ANADYS PHARMACEUTICALS, INC.

(21) Appl. No.: 10/434,842

(22) Filed: May 9, 2003

Related U.S. Application Data
(63) Continuation of application No. 10/233,307, filed on Aug. 28, 2002, now abandoned.
Application No. 10/233,307 is a continuation-in-part of application No. 10/005,469, filed on Nov. 7, 2001.
(60) Provisional application No. 60/245,866, filed on Nov. 7, 2000.

(51) Int. Cl. C12Q 1/70; C07H 21/04; C12N 7/00; C12N 5/08

(52) U.S. Cl. 435/5; 435/235.1; 435/370; 536/23.72

(57) ABSTRACT

The present invention relates to recombinant hepatitis C virus (HCV)-derived nucleic acids and to stable cell clones derived from human hepatoma Huh-7 cell line supporting high titer replication of said recombinant HCV nucleic acids. The subgenomic HCV replicons and cell clones of the instant invention represent the in vitro system of choice for studies of HCV propagation, anti-viral drug screening, and vaccine development.

This application claims benefit of priority to U.S. application Ser. No. 10/005,469, entitled “Hepatitis C Virus Constructs Characterized by High Efficiency Replication” filed Nov. 7, 2001, and to U.S. Provisional Application No. 60/245,866 filed Nov. 7, 2000, both of which are hereby incorporated by reference in their entireties.

### FIELD OF THE INVENTION

The present invention relates to recombinant hepatitis C virus (HCV)-derived nucleic acids and to stable rapidly growing cell clones supporting their efficient replication.

### BACKGROUND OF THE INVENTION

Infection by hepatitis C virus (HCV) is a compelling human medical problem. HCV is recognized as the causative agent for most cases of non-A and non-B hepatitis, with an estimated worldwide prevalence of 170 million cases (i.e., 2-3%) (Choo et al., Science, 1989, 244:359-362; Kuo et al., Science, 1989, 244:362-364; Purcell, FEMS Microbiol. Rev., 1994, 14:181-192; Van der Poel, In: Current Studies in Hematology and Blood Transfusion, Reesink ed., Basel: Karger, pp. 137-163, 1994). Four million individuals may be infected in the United States alone (Alter and Mast, Gastroenterol. Clin. North Am., 1994, 23:437-455).

HCV is primarily transmitted parenterally, although sexual and perinatal transmission do appear to occur. At present, no risk factor has been identified in about 40% of HCV-infected individuals in the US (Alter, Infect. Agents Dis., 1993, 2:155-166). Upon first exposure to HCV, only about 10% or less of infected individuals develop acute clinical hepatitis, while others appear to resolve the infection spontaneously. In most instances, however, the virus-establishes a chronic infection that persists for decades, leading in about 50% of all cases to chronic hepatitis, which can, in turn, develop into liver cirrhosis and/or hepatocellular carcinoma (Iwarson, FEMS Microbiol. Rev., 1994, 14:201-204; Kew, ibid. pp.211-220; Saito et al., Proc. Natl. Acad. Sci. USA, 1990, 87:6547-6549).

Apart from liver cells, HCV can also replicate in peripheral blood mononuclear cells (PBMCs) both iii vivo and in experimentally infected B- and T-cell lines (U.S. Pat. Nos. 5,679,342 and 5,968,775). Such a lymphotropism may account for the numerous immunological disorders, in particular type II and type III cryoglobulinaemia, observed in more than 50% of chronic hepatitis C patients (Esteban et al., In: Hepatitis C Virus, Reesink ed., Basel: Karge, 1998, pp. 102-118).

### HCV Structure and Genome Organization

Given the high prevalence of the virus, HCV has become a focus of intensive research (for recent review see Bartenschlager and Lohmann, J. Gen. Virol., 2000, 81:1631-1648; Rosen and Gretch, Mol. Medicine Today, 1999, 5: 393-399). Originally cloned in 1989 (Choo et al., supra), the viral genome is now well characterized. HCV is a (+) strand enveloped RNA virus, i.e. its genome is represented by a coding single stranded RNA (cRNA) which is packaged with the structural proteins in a viral particle surrounded by a host cell-derived membrane. HCV has been classified as the sole member of a distinct genus called hepacivirus in the family Flaviviridae, which includes, e.g., the flaviviruses and the animal pathogenic pestiviruses. Its genome has a length of approximately 9.6 kb and its single, long open reading frame (ORF) encodes an approximately 3000-amino acid polyprotein that is proteolytically cleaved into a set of distinct products (FIG. 1 [prior art]; see also Rice, In: Virology, Fields et al. eds., Lippincott-Raven, 1996, Vol.1, pp.931-960; Clarke, J. Gen. Virol., 1997, 78:2397).

The HCV ORF is flanked at the 5′ and 3′ ends by nontranslated regions (NTRs). Translation of the ORF is directed via an approximately 340 nucleotide (nt) long 5′ NTR functioning as an internal ribosome entry site (IRES) and permitting the direct binding of ribosomes in close proximity to the start codon of the ORF (Tsukiyama-Kohara et al., J. Virol., 1992, 66:1476-1483; Wang et al., J. Virol., 1993, 67:3338-3344). The first approximately 40 nucleotides of the 5′ NTR are not required for translation but, based on analogy with other (+) strand RNA viruses, are involved most likely in RNA replication (Boyer and Haenni, Virology, 1994, 198:415-426). The 3′ NTR has a tripartite structure composed of a variable sequence following the stop codon of the ORF, a poly(U) tract of heterogeneous length and a highly conserved 98 nucleotide sequence essential for replication in vivo (Kolykhalov et al., J. Virol., 1996, 70:3363; Tanaka et al., Biochem. Biophys. Res. Comm., 1995, 215:744; Tanaka et al., J. Virol., 1996, 70:3307; Yamada et al., Virology, 1996, 223:255; Yanagi et al., Proc. Natl. Acad. Sci. U.S.A., 1999, 96:2291; Kolykhalov et al., J. Virol., 2000, 74:2046-2051).

The HCV polyprotein is cleaved co- and post-translationally by cellular and viral proteinases into ten different products, with the structural proteins located in the N-terminal one-third and the non-structural (NS) proteins (i.e., proteins which are not expected to be constituents of the virus particle) in the remainder (FIG. 1; reviewed in Bartenschlager and Lohmann, supra; Bartenschlager, J. Viral Hepatitis, 1999, 6:165-181; Reed and Rice, In: Hepatitis C Virus, Reesink ed., Basel: Karger, 1998, pp. 1-37). The first cleavage product of the polyprotein is a highly basic core protein, which is the major constituent of the nucleocapsid (Yasui et al., J. Virol., 1998, 72:6048-605) and is involved in modulation of several cellular processes leading to induction of hepatocellular carcinoma (Chang et al., J. Virol., 1998, 72: 3060-3065; Chen et al., J. Virol., 1997, 71:9417-9426; Matsumoto et al., ibid., pp.1301-1309; Moriya et al., Nature Med., 1998, 4:1065-1067). Envelope proteins E1 and E2 are highly glycosylated type I transmembrane proteins, forming two types of stable heterodimeric complexes (Deleersnyder et al., J. Virol., 1997, 71:697-704). In addition, E2 was shown to interact with the interferon (IFN)-induced double-stranded RNA-activated protein kinase PKR, allowing continuation of translation of HCV RNA in the presence of IFN (Taylor et al., Science, 1999, 285:107-110). Protein p7, located at the C-terminus of E2, is a highly hydrophobic polypeptide of unknown function. Most of the nonstructural proteins NS2-5B are required for replication of the viral RNA (Lohmann et al., Science, 1999, 285:110-113). NS2 and the N-terminal domain of NS3 constitute the NS2-3 proteinase, catalysing cleavage at the NS2/3 site (Grakoui et al., 1993, Proc. Natl. Acad. Sci. USA, 1993, 90:10583-10587; Hijikata et al., J. Virol., 1993, 67:4665-4675; Hirowatari et al., Arch. Virol., 1993, 133:349-356). NS3 is a bifunctional molecule carrying, in the N-terminal approximately 180 residues, a serine-type proteinase responsible for cleavage at the NS3/4A, NS4A/B, NS4B/5A and NS5A/B sites and, in the C-terminal remainder, NTPase/helicase activities essential for translation and replication of the HCV genome (Bartenschlager et al., J. Virol., 1993, 67:3835-3844; Eckart et al., Biochem. Biophys. Res. Comm., 1993, 192:399-406; Grakoui et al., J. Virol., 1993, 67:2832-2843; Gwack et al., Biochem. Biophys. Res. Comm., 1996, 225:654-659; Hong et al., J. Virol., 1996, 70:4261-4268; Kim et al., Biochem. Biophys. Res. Comm., 1995, 215:160-166; Suzich et al., J. Virol., 1993, 67:6152-6158; Tai et al., J. Virol., 1996, 70:8477-8484; Tomei et al., J. Gen. Virol., 1993, 77:1065-1070; Kolykhalov et al., 2000, supra). In addition, NS3 may interfere with host cell functions by inhibiting protein kinase A (PKA)-mediated signal transduction and/or by inducing cell transformation (Borowski et al., Eur. J. Biochem., 1996, 237:611-618; Sakamuro et al., J. Virol., 1995, 69:3893-3896). NS4A is an essential cofactor of the NS3 proteinase and is required for efficient polyprotein processing (Bartenschlager et al., J. Virol., 1994, 68:5045-5055; Failla et al., ibid., pp. 3753-3760; Lin et al., ibid., pp. 8147-8157; Tanji et al., J. Virol., 1995, 69:1575-1581). The function of the hydrophobic NS4B is so far unknown. NS5A is a highly phosphorylated protein (Asabe et al., J. Virol., 1997, 71:790-796; Kaneko et al., Biochem. Biophys. Res. Comm., 1994, 205:320-326; Koch and Bartenschlager, J. Virol., 1999, 73:7138-7146; Neddermann et al., ibid., pp. 9984-9991; Tanji et al., J. Virol., 1995, 69:3980-3986) which appears to interfere with the antiviral effect of IFN by binding to PKR (Gale et al., Virology, 1997, 230:217-227; Gale et al., Mol. Cell Biol., 1998, 18:5208-5218) and may also play role in RNA replication. NS5B was identified as the RNA-dependent RNA polymerase (RdRp) (Al et al., Virus Res., 1998, 53:141-149; Behrens et al., EMBO J., 1996, 15:12-22; Lohmann et al., J. Virol., 1997, 71:8416-8428; Yamashita et al., J. Biol. Chem., 1998, 273:15479-15486; Yuan et al., Biochem. Biophys. Res. Comm., 1997, 232:231-235).

### HCV Replication and Variability

Similarly to related positive (+) strand RNA viruses, HCV replication occurs by means of a negative (−) strand RNA intermediate and is catalyzed by the NS proteins forming a cytoplasmic membrane-associated replicase complex. HCV replication cycle can be summarized as follows (FIG. 2 [prior art]): (1) penetration of the host cell and liberation of the genomic RNA (cRNA) from the virus particle into the cytoplasm; (2) translation of the input cRNA, processing of the polyprotein and formation of a replicase complex associated with intracellular membranes; (3) utilization of the input (+) strand for synthesis of a (−) strand RNA intermediate; (4) production of new (+) strand RNA molecules which can be used for synthesis of new (−) strands, for polyprotein expression or for packaging into progeny virions; (5) release of virus from the infected cell via cellular secretion pathway resulting in formation of cell-derived viral membrane envelope.

The dynamics of HCV replication can be deduced from the rapid rates of virus production and emergence of mutants. Analysis of viral dynamics during antiviral treatment of patients with IFN-α revealed a virion half-life of 3-5 hours and a clearance and production rate of approximately 10¹² particles per day (Zeuzem et al., Hepatology, 1998, 28:245-252; Neumann et al., Science, 1998, 282:103-107; Ramratnam et al., Lancet, 1999, 354:1782-1785). Although in absolute amounts these numbers are high, they are not with respect to a single cell, corresponding to a virion production rate of 50 particles per hepatocyte per day (Neumann et al., supra).

Another feature of HCV replication is a rapid generation of virus variants. Early studies of the mutation rate of HCV in chronically infected humans and chimpanzees demonstrated that this virus mutated very rapidly with the rate of change varying between different genomic regions (Ogata et al., Proc. Natl. Acad. Sci. USA, 1991, 88:3392-3396; Okamoto et al., Virology, 1992, 190:894-899). Thus, the highest mutation rate was found in the E1 and E2 genes with especially high rate observed in a short sequence encoding the domain located at the N-terminus of E2 protein (hence termed “hypervariable region 1” or “HVR1”). Accordingly, even within a single patient HCV does not exist as a single entity but rather as a collection of microvariants of a predominant “master sequence”, a phenomenon that has been referred to as quasispecies (reviewed in Bukh et al., Semin. Liv. Dis., 1995, 15:41-63; Bukh et al., Clin. Exp. Rheumatol., 1995, 13(suppl.):S3-S7; Holland et al., Curr. Topics Microbiol. Immunol., 176:1-20). The master sequence, as well as the consensus sequence of the quasispecies sequence population have been found to change sequentially during the infection. The production of such large number of variants is primarily due to the high error rate of the viral RdRp that, based on analogies with RdRps of other (+) strand RNA viruses, is expected to be in the range of 10⁻⁴. Using comparative sequence analyses of HCV genomes isolated over intervals of 8 or 13 years, a mutation rate of 1.44×10⁻³ or 1.92×10⁻³ base substitutions per site per year was found, respectively (Ogata et al., supra; Okamoto et al., supra). The high variation observed with HCV replication may also account for the fact that a significant fraction of virus genomes appear to be defective (Martell et al., J. Virol., 1992, 66:3225-3229).

The biological consequences of quasispecies include: (i) the development of escape mutants to humoral and cellular immunity leading to the establishment of a persistent infection; (ii) variable cell tropism (e.g., lymphotropic vs hepatotropic); (iii) vaccine failure, and (iv) rapid development of drug resistance (Bukh et al., Semin. Liv. Dis., supra). For example, it has been found that the HVR1 contains epitopes that elicit a specific humoral immune response and that sequential changes of HVR1 during infection resulted in the emergence of epitopes that were not recognized by pre-existing antibodies (Weiner et al., Proc. Natl. Acad. Sci. USA, 1992, 89:3468-72; Taniguchi et al., Virology, 1993, 195:297-301; Kato et al., J. Virol., 1993, 67:3923-30; Kato et al., J. Virol., 1994, 68:4776-84).

It is now well established that HCV exists as distinct genotypes among different HCV isolates with prevalence of each of the genotypes in specific geographical locations. Based on the genomic variability in the most highly conserved NS5B and E1 sequences, HCV has been classified into at least 9 major genetic groups (genotypes 1a, 1b, 1c, 2a, 2b, 3a, 7, 8, 9) with total over 30 subtypes (Bukh et al., Clin. Exp. Rheumatol., supra; Simmonds et al., J. Gen. Virol., 1993, 74:2391-2399). Several recent studies indicate that the extensive genetic heterogeneity of HCV may have important clinical implications, with genotype 1b (prevalent in the US and Europe) being associated with a more severe liver disease and a poorer response to interferon therapy (reviewed in Bukh et al., supra). Anti-HCV Therapies

Despite the intense research, the only anti-HCV therapy currently available is administration of a high dose of IFN-α or a combination treatment with IFN-α and the nucleoside analogue ribavirin. However, only about 40% of all patients benefit from this treatment and develop a sustained response, demonstrating the urgent need for more effective antiviral therapeutics (Marcellin et al., Ann. Intern. Med., 1997, 127:875-881; Reichard et al., Lancet, 1998, 351:83-87).

As mentioned above, anti-HCV vaccine development has been hampered by the high degree of viral variability leading to efficient immune evasion and the lack of protection against reinfection, even with the same inoculum (Farci et al., Science, 1992, 258:135-140; Kao et al., J. Med. Virol., 1996, 50:303-308; Shimizu et al., 1994, J. Virol., 68:1494-1500; Wyatt et al., J. Virol., 1998, 72:1725-1730). Among the most promising approaches to vaccine development, is immunization with highly conserved HCV core protein alone or in combination with E1 and/or E2 envelope proteins, and/or NS3 protein (Major et al., 1995, J. Virol., 69:5798-5805; Tokushige et al., Hepatology, 1996, 24:14-20; Geissler et al., J. Immunol., 1997, 158:1231-1237; Inchauspe et al., Vaccine, 1997, 15:853-856).

In view of the reasonable scepticism over the “universal” anti-HCV vaccine, the importance of alternative therapies increases. Such therapies include without limitation: (i) small molecule inhibitors directed against specific viral targets (e.g., E1/E2 envelope proteins or NS3 protease/helicase); (ii) antisense oligonucleotides and ribozymes for the inhibition of HCV replication, and (iii) transdominant-negative proteins (Kim et al., Cell, 1996, 87:343-355; Love et al., ibid., 331-342; Yao et al., Nat. Struct. Biol., 1997, 4:463-467; Yan et al., Protein Sci., 1998, 7:837-847; Von Wizsaker et al., Hepatology, 1997, 26:251-255; Lieber et al., J. Virol., 1996, 70:8782-8791; Rosen and Gretch, supra; Saito et al., Gastroenterology, 1997, 112:1321-1330; Nakano et al., J. Virol., 1997, 71:7101-7109; Fournillier et al., Hepatology, 1998, 28:237-244; Wakita et al., J. Biol. Chem., 1994, 269:14205-14210; Mizutani et al., Biochem. Biophys. Res. Comm., 1995, 212:906-911; Alt et al., Hepatology, 1995, 22:707-717; Havecak et al., J. Virol., 1996, 70:5203-5212; Lima et al., J. Biol. Chem., 1997, 272:626-638; Alt et al., Arch. Virol., 1997, 142:589-599; Wu and Wu, Gastroenterology, 1998, 114:1304-1312; Sakamoto et al., J. Clin. Invest., 1996, 98:2720-2728; Ruiz et al., J. Viral Hepatitis, 1999, 6:17-34).

### In Vivo and In Vitro Systems for HCV Infection

As the currently available chimpanzee models are too expensive to be practical for early stage evaluation of anti-HCV therapeutics, there is a need in the art to develop more manageable and efficient in vivo and in vitro model systems for HCV infection.

In an attempt to establish a small animal model Xie et al. (Virology, 1998, 244:513-520) experimentally inoculated Tupaias (T. belangeri chinensis), a tree shrew species which adapt and breed in the laboratory environment, are closely related to primates, and were previously shown to be susceptible to infection with the human rotavirus (Wan et al., Natl. Med. J. Chin., 1982, 62:461-465), herpes simplex virus type 1 and 2 (Darai et al., J. Infect. Dis., 1978, 137:221-226), and human hepatitis viruses A, B (both in vitro and in vivo), and Delta (Li et al., Chung. Hua. I. Hsuch. Tsa. Chih., 1995, 75:611-613; Walter et al., Hepatology, 1996, 24:1-5; Yan et al., J. Cancer Res. Clin. Oncol., 1996, 122:283-288 and 289-295; Zan et al., Acta Acad. Med. Sin., 1981, 3:148-152). Although, upon inoculation, only about one-quarter of the animals became infected with HCV and developed either transient or intermittent viraemia with rather low titers, the Tupaia animal model appears to be very promising. Recently, two potential alternatives have been described for the propagation of hepatitis B viruses that might be used for HCV as well. Both systems are based on the engraftment of human liver tissue into immuno-compromised mice (Ilan et al., Hepatology, 1999, 29:553-562; Petersen et al., Proc. Natl Acad. Sci. USA, 1998, 95:310-315).

Although development of a small animal model is critical for studies of HCV pathophysiology and for assaying toxicology and pharmacokinetics of anti-HCV therapeutics, it is of primary importance to create a convenient and reliable cell culture-based assay system that supports HCV infection and replication and allows detailed molecular studies of HCV propagation and efficient high-throughput evaluation of anti-HCV therapeutics.

Until recently, in vitro research on HCV has depended largely on (i) analogies to the closely related flavi- and pestiviruses, (ii) characterization of recombinantly produced HCV proteins, (iii) infection of primary cell cultures with HCV-containing sera of infected individuals, and (iv) cultivation of primary cells derived from chronically infected tissues (Lanford et al., 1994, Virology, 202:606; Shimizu et al., Proc. Natl. Acad. Sci. U.S.A., 1992, 89:5477; Mizutani et al., J. Virol., 1996, 70:7219-7223; Ikeda et al., Virus Res., 1998, 56:157; Fournier et al., J. Gen. Virol., 1998, 79:2376). For example, HCV replication was reported in primary hepatocytes from humans or chimpanzees following infection with high titer HCV-containing serum (Fournier et al., supra; Iacovacci et al., Res. Virol., 1993, 144:275-279; Lanford et al., supra; Rumin et al., J. Gen. Virol., 1999, 80:3007-3018) and in PBMCs isolated from chronically infected patients (Bouffard et al., J. Infect. Diseases, 1992, 166:1276-1280; Müller et al., J. Gen. Virol., 1993, 74:669-676; Zignego et al., J. Hepatol., 1992, 15:382-386).

In the primary cell cultures HCV replication was detected using a number of highly sensitive methods: (i) detection of (−) strand replicative RNA intermediates by strand-specific reverse transcriptase-polymerase chain reaction (RT-PCR) or Nothern blot; (ii) determination of an increase of (+) strand RNA during the cultivation period using either b-DNA assay or quantitative RT-PCR; (iii) detection of inhibition of replication upon incubation of the cells with IFN-α or antisense oligonucleotides; (iv) the ability of the cell culture medium (containing viral particles) to cause infection upon addition to naive cells; (v) sequence analysis of HCV genomes or genome fragments to demonstrate genomic variability and selection of variants upon infection and cultivation, and (vi) detection of viral antigens in situ or upon isolation using, e.g., immunofluorescence, Western blotting, or flow cytometry.

It should be noted, however, that primary cell cultures are not routinely available, are hard to maintain, and suffer from poor reproducibility and a low level of HCV replication that can be measured only with highly sensitive techniques. Accordingly, numerous attempts have been made to develop stable HCV-replicating cell lines. Initially, these cell lines were also developed by infecting them with the virus isolated from infected individuals. Such HCV-infected cell lines, secondary monkey kidney cells CV-1 and human diploid fibroblasts VERO, are disclosed, for example, in the PCT Application No. WO 96/24662. With respect to hepatoma cell lines, the most detailed results are available for the non-neoplastic cell line PH5CH (Ikeda et al., 1998, supra; Kato et al., Jap. J. Cancer Res., 1996, 87:787-792). However, a strong selection for HCV variants in the HVR1 of the E2 protein was observed in this cell line suggesting that only certain variants can bind to or replicate in these cells. A similar decrease of complexity of viral quasispecies has been described for HCV propagated in primary human hepatocytes for up to 3 months (Rumin et al., supra).

HCV replication upon infection was also demonstrated in cultured cells derived from the T-cell lines MT-2, HPBMa10-2, and MOLT-4 and from the B-cell line Daudi. For example, in HPBMa10-2 and Daudi cells, long-term propagation of HCV for more than 1 year has been described (Nakajima et al., J. Virol., 1996, 70:3325-3329), and virus could be transmitted several times to naive cells by cocultivation (Shimizu and Yoshikura, J. Virol., 1994, 68:8406-8408). HCV-replicating B- and T-cell lines are also disclosed in the U.S. Pat. No. 5,679,342. However, similarly to PH5CH cells, it was found that only certain virus variants replicate in HPBMa10-2 and Daudi cells as well as in MT-2C cells, suggesting the selection of lymphotropic HCV quasispecies (Mizutani et al., J. Virol., 1996, supra; Sugiyama et al., J. Gen. Virol., 1997, 78:329-336).

### Attempts to Create Stable Cell Lines Containing Self-Propagating Recombinant HCV Replicons

The recent construction of cloned HCV genomes and demonstration of their ability to replicate and cause disease development after intrahepatic inoculation of chimpanzees (Beard et al., Hepatology, 1999, 30:316-324; Kolykhalov et al., Science, 1997, 277:570-574; Yanagi et al., Proc. Natl. Acad. Sci. USA, 1997, 94:8738; Yanagi et al., Virology, 1998, 244:161) has opened some new avenues to study HCV replication and pathogenesis. Specifically, it made feasible the development of stable cell cultures containing selectable HCV replicons. Compared to the infection of cell lines with HCV-containing patient material, the introduction of cloned virus genomes is superior because the inoculum is well defined and can be generated in high quantities. Most importantly, the genome can be manipulated at will, permitting a detailed genetic analysis of viral functions leading to successful development of anti-viral therapeutics.

Similarly to experience with several (+) strand RNA viruses (Boyer and Haenni, supra), it became clear with the first attempts to create self-replicating subgenomic HCV clones in culture that the use of in vitro synthesized RNA transcripts (cRNA) of defined structure (produced, e.g., using T7 or SP6 in vitro transcription system) is advantageous to transfection of DNA constructs. Indeed, direct transfection of cRNA avoids the involvement of the cell nucleus and therefore potential problems associated with transcriptional regulation, splicing, incorrect 5′ and 3′-end processing, and nucleo-cytoplasmic transport (Dash et al., Am J. Pathol., 1997, 151:363-373).

However, in contrast to many other (+) strand RNA viruses, construction of self-replicating subgenomic HCV clones in culture turned out to be very difficult. Until recently, only a few successful attempts were reported, each lacking important controls and/or being somewhat controversial. Thus, Baumert et al. (J. Virol., 1998, 72:3827-3836) described assembly of poorly characterized HCV-like particles in insect cells upon introduction of a recombinant baculovirus containing the cDNA encoding HCV ORF. Dash et al. (supra) and Yoo et al. (J. Virol., 1995, 69:32-38) reported successful replication of putative HCV cRNA upon transfection in the human hepatoma cell lines Huh-7 and HepG2, respectively. However, both studies appear to be highly questionable as they describe the propagation of truncated HCV genomes lacking the authentic 3′ NTR, which is essential for replication in vivo (Yanagi et al., 1999, supra; Kolykhalov et al., 2000, supra).

The most convincing evidence of a functional in vitro cell-based system for replication of recombinant HCV came from a recent report by Lohmann et al. (Science, 1999, 285:110-113). These authors have described selectable subgenomic HCV RNA molecules replicating after transfection into the human hepatoma cell line Huh-7. Similar subgenomic HCV replicons capable of propagating in tissue culture are disclosed in PCT Application No. WO 98/39031. Based on the assumption that high expression levels of the structural proteins might be cytotoxic (Moradpour et al., Biochem. Biophys. Res. Comm., 1998, 246:920-924) and the observation that for several (+) strand RNA viruses (e.g., alpha-, flavi- and pestiviruses) the structural proteins are not required for RNA replication (Behrens et al., J. Virol., 1998, 72:2364-2372; Khromykh and Westaway, J. Virol., 1997, 71:1497-1505; Liljestrom and Garoff, Biotechnol., 1991, 9:1356-1361), Lohmann et al. deleted the sequences of the structural proteins in their HCV-derived constructs. In addition, to allow selection for only those cells which support efficient HCV replication, the gene encoding the neomycin phosphotransferase (neo) and conferring resistance to the antibiotic G418, was introduced downstream of the HCV IRES (FIG. 3). A second IRES element was included to allow translation of the HCV NS proteins. Upon transfection of these bicistronic RNAs and selection in the presence of G418, only cells supporting replication of HCV-derived RNAs amplified the neo gene and developed resistance, whereas non-transfected cells and cells unable to support replication died. The selected cells carried large amounts of HCV RNAs detectable by Northern blot, or after metabolic radiolabeling with ³[H]uridine, providing formal proof that these RNAs were actively replicating in the cells. As expected for a replicative intermediate, (−) strand RNA was present in approximately 10-fold lower amounts compared to (+) strand RNA. Finally, HCV proteins could be detected by immunoprecipitation after metabolic radiolabelling with ³⁵[S]methionine or Western blot and were confined to the cytoplasm (see also Bartenschlager and Lohmann, 2000, supra).

Although replicon titers observed in several clones generated by Lohmann et al. were several orders of magnitude higher compared to previously available cell culture-based HCV infection systems (described above, see also Blight and Gowans, Viral Hepatitis Rev., 1995, 1:143-155), the overall efficiency of clone generation upon transfection was very low. Taken together with the observation that the majority of cell clones containing replication-competent subgenomic HCV RNAs were growing much slower than the naive Huh-7 cells or the cells transformed with replication-deficient RNA, it can be concluded that the recombinant HCV replicons of Lohmann et al. are toxic to their host cells. Clearly, such cytotoxic replicons are deficient as an in vitro model of HCV propagation as they cannot attain their maximal replication titers and therefore cannot provide sufficient amounts of subgenomic viral nucleic acids required for generation of recombinant anti-viral vaccines, for studies of some of the viral processes, and for sensitive large-scale anti-viral drug screening.

In view of the above, despite the progress made in the last several years, the field is still lacking an efficient stable cell culture system for high titer propagation of recombinant HCV. Such a system is needed for (i) further studies of intracellular viral processes (e.g., analysis of HCV receptor binding, cellular infection, replication, virion assembly, and release); (ii) generation and testing of anti-viral vaccines; (iii) screening and testing of anti-viral drugs; (iv) development of targets and methods for HCV diagnostics, and (v) production of concentrated virion and protein stocks (e.g., for structural analysis of virion components leading to epitope determination for immunotherapy).

The present invention addresses these and other needs in the art by providing novel mutated recombinant HCV-derived nucleic acids and novel rapidly growing “adapted” cell clones supporting their efficient replication.

### SUMMARY OF THE INVENTION

The present invention provides novel recombinant hepatitis C(HCV)-derived nucleic acids. Preferred subgenomic HCV replicons of the invention include HCVR 2 (SEQ ID NO: 2), HVCR 8 (SEQ ID NO: 3), HCVR 9 (SEQ ID NO: 4), HCVR 22 (SEQ ID NO: 5), HCVR 24 (SEQ ID NO: 6), FCA1 (SEQ ID NO: 15), FCA4 (SEQ ID NO: 16), and FCA22 (SEQ ID NO: 17). These replicons are derived from the parental HCV genotype 1b-based recombinant clone I377-NS3-3′UTR (SEQ ID NO: 1) and contain one or more nucleotide changes (e.g., as shown in Table I and Table II, see Examples 1 and 2) which occurred following their prolonged replication in the Huh-7-derived cell clones of the present invention under stringent selection conditions. At least some of these mutations are indicative of the non-structural HCV genome regions which are responsible for high-titer viral replication and virus-induced cytotoxicity.

In a specific embodiment, the present invention is directed to plasmid clones which can be transcribed to produce self-replicating recombinant HCV RNAs of the invention.

In a separate embodiment, the instant invention includes a method for generating novel efficiently replicating recombinant HCV-derived nucleic acids containing the critical elements of the HCV replicons described above and comprising from 5′ to 3′ on the positive-sense nucleic acid (1) a functional 5′ non-translated region (NTR) comprising an extreme 5′-terminal conserved sequence, (2) at least one open reading frame (ORF) encoding a heterologous gene operatively associated with an expression control sequence, wherein the heterologous gene and expression control sequence are oriented on the positive-strand nucleic acid molecule, (3) an ORF encoding at least a portion of an HCV polyprotein whose cleavage products form functional components of HCV virus particles and RNA replication machinery, and (4) a 3′ NTR comprising an extreme 3′-terminal conserved sequence. In a specific embodiment, where the selection marker ORF is a drug resistance gene, this gene is a neomycin resistance gene (neo) operatively associated with an internal ribosome entry site (IRES).

The instant invention also includes a method for propagating the disclosed recombinant HCV-derived nucleic acids in vitro by culturing a cell line transfected or infected with an appropriate amount of HCV RNA, e.g., as produced from the plasmid clones recited above, under conditions that permit replication of the HCV RNA. In a specific embodiment, replication of the disclosed novel HCV-derived nucleic acids in susceptible cultured cells leads to the generation of potentially infectious recombinant viral particles which can be used as an attenuated anti-HCV vaccine.

Accordingly, in conjunction with the recombinant nucleic acids disclosed herein, the present invention advantageously provides cell lines, which are susceptible to HCV infection and/or transfection and support replication of such recombinant nucleic acids. In a preferred embodiment, the susceptible cell line of the invention is a human hepatoma cell line Huh-7.

The invention further provides stable “adapted” cell clones which are derived from Huh-7 cell line and are characterized by the growth properties which are similar to or indistinguishable from the naive (i.e., untransfected) Huh-7 cells (e.g., as shown in FIG. 5). The invention also provides “adapted” cell clones which are derived from the Huh-7 cell line after transfection with a subgenomic HCV RNA that contain a subgenomic HCV replicon that has a greater efficiency of colony formation than subgenomic HCV replicon I377/NS3-3′. Some of the most efficiently propagating cell clones of the present invention, i.e., HCVR 2, 8, 9, 22 and 24, were deposited with the American Type Culture Collection (ATCC), 10801 University Boulevard, Manassas, Va. 20110-2209, USA on Sep. 20, 2000, and assigned Accession Nos. PTA-2489, PTA-2490, PTA-2486, PTA-2487, and PTA-2488, respectively. Other cell clones of the present invention, i.e., FCA1, FCA4, and FCA22, were deposited with the American Type Culture Collection (ATCC), 10801 University Boulevard, Manassas, Va. 20110-2209, USA on ______ [PLEASE PROVIDE DATE OF DEPOSIT], and assigned Accession Nos. ______ [PLEASE PROVIDE ATCC ACCESSION NOS.], respectively.

The materials disclosed herein provide methods for screening (both in vitro and in vivo) for agents capable of modulating HCV infection and/or replication and/or virion assembly. Such methods include administering a candidate agent to HCV-replicating cell line(s) of the invention, and testing for an increase or decrease in a level of subgenomic HCV replication or HCV-associated protein expression compared to a level of HCV replication or HCV-associated protein expression in a control cell line transfected with replication-defective construct or in the same cell line prior to administration of the candidate agent, wherein a decrease in the level of HCV replication or HCV-associated protein expression is indicative of the inhibitory activity of the agent.

In a specific embodiment, HCV-replicating cell lines of the present invention provide a convenient system for high-throughput initial screening of potential anti-HCV therapeutics.

Further provided herein is a method for generating massive quantities of recombinant HCV replicons (which can serve as a basis for anti-HCV vaccine development) from the cell clones of the present invention, said replicons being produced either as intracellular nucleic acids or as infectious or non-infectious recombinant viral particles.

The present invention also has significant diagnostic implications. For example, the invention provides an in vitro method for detecting antibodies to HCV in a biological sample from a subject comprising contacting the sample with HCV-replicating cells, cellular fractions, isolated HCV-derived proteins, or HCV-derived viral particles prepared as described above. The contacting operation is conducted under conditions that permit binding of HCV-specific antibodies in the sample to the HCV protein(s); and detecting binding of antibodies in the sample to the HCV-derived protein(s). Detecting binding of antibodies in the sample to the HCV protein(s) is indicative of the presence of HCV infection in the subject from which the sample was derived.

In summary, the present invention provides nucleic acids encoding recombinant HCV replicons, which are capable of efficient propagation and expression of HCV-derived proteins in a cell culture system.

The invention further provides susceptible cell lines (and “adapted” cell clones derived from them), which support high titer replication of recombinant HCV-derived nucleic acids.

By providing cell clones supporting efficient subgenomic HCV replication, the present invention provides (i) in vitro cell culture models of HCV propagation; (ii) systems for screening candidate anti-viral compounds and evaluating drug resistance; (iii) methods for diagnosing HCV infection, and (iv) systems for production of large quantities of HCV-derived components or recombinant viral particles for antibody generation and/or vaccine development.

The present invention meets these and other objects of the invention, as set forth in greater detail in the Detailed Description and Examples, including the accompanying Drawings.

### BRIEF DESCRIPTION OF THE DRAWINGS

FIG. 1 (prior art) depicts HCV genome structure and polyprotein processing. At the top is a schematic representation of the viral genome with the structural and nonstructural protein coding regions, and the 5′ and 3′ NTRs. Boxes below the genome identify proteins generated by the proteolytic processing cascade. Different types of arrows represent different types of proteases responsible for polyprotein processing at each particular site.

FIG. 2 (prior art) is a schematic representation of the HCV life cycle comprising the following stages: (1) penetration of the host cell and liberation of the (+) strand genomic RNA (cRNA) from the virus particle into the cytoplasm; (2) translation of the input cRNA, processing of the polyprotein and formation of a replicase complex associated with intracellular membranes; (3) utilization of the input (+) strand for synthesis of a (−) strand RNA intermediate; (4) production of new (+) strand RNA molecules which can be used for synthesis of new (−) strands, for polyprotein expression or for packaging into progeny virions; (5) release of virus from the infected cell via cellular secretion pathway resulting in formation of cell-derived viral membrane envelope.

FIG. 3 is a schematic representation of the recombinant parental HCV replicon I377/NS3-3′UTR (SEQ ID NO: 1) composed of the 5′HCV-IRES, the neo gene, the EMCV-IRES, and HCV sequences from NS3 up to the authentic 3′ end.

FIG. 4 depicts detection of (+) strand HCV RNA in subpassaged Huh-7 cell clones by RT-PCR using primers flanking the region corresponding to NS5B nt 7435-7750. Shown is 2% agarose gel electrophoresis analysis of RT-PCR fragments obtained by amplifying total RNA isolated from G418-resistant Huh-7 cell clones HCVR 2, 6, 8, 9 and 12 (lanes 2-6). As a negative control, PCR was performed without a DNA template (lane 1). Lane MW shows position of molecular size markers.

FIG. 5 is a graphic representation of relative growth rates of Huh-7-derived “adapted” cell clones supporting efficient subgenomic HCV replication. Growth rates (shown as a function of optical density after sulphorhodamine staining for total cellular proteins) of clones HCVR 2, 8, 9, 22 and 24 are compared to the growth rate of naïve non-transfected Huh-7 cells (black line) at 9 weeks post-transfection.

FIG. 6 depicts the replication of subgenomic replicons in Huh7 cells.

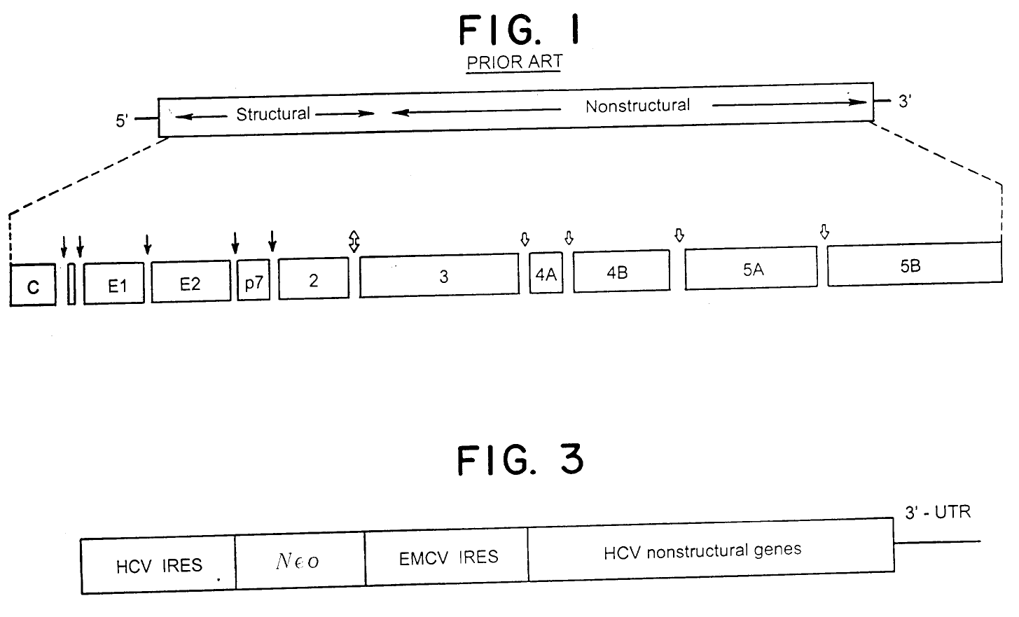

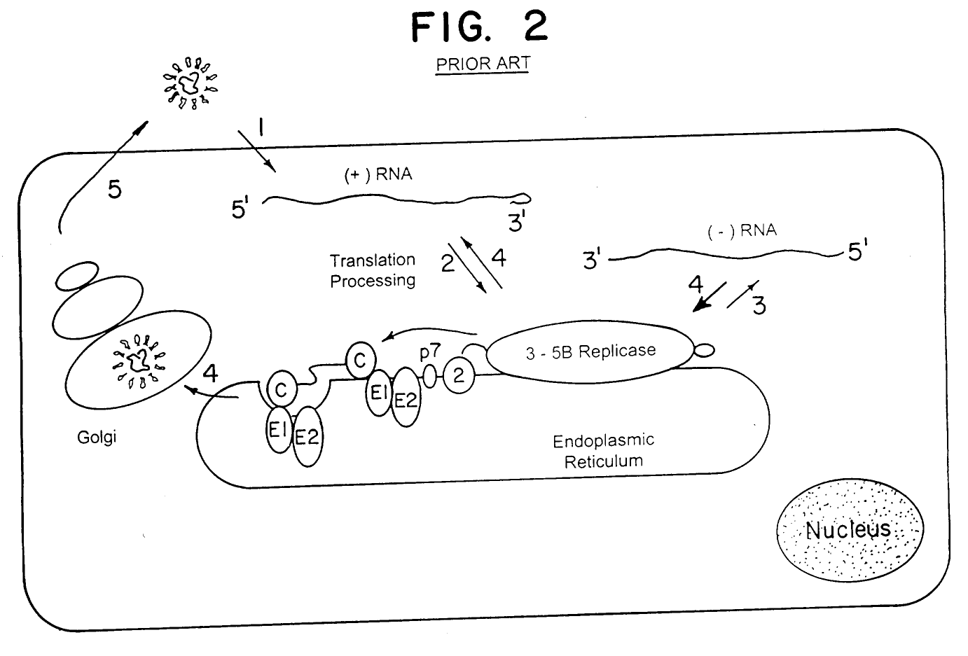

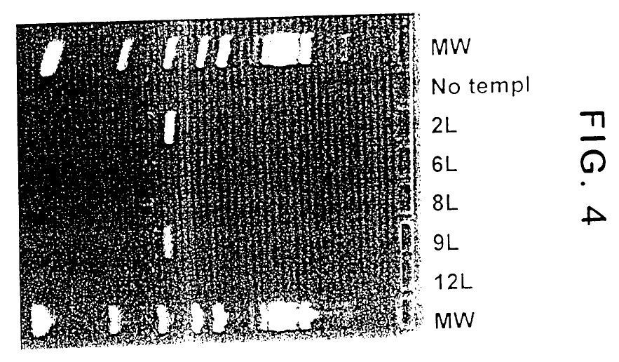

(A) shows colonies of Huh 7 cells resulting from transfection with in vitro-transcribed RNA of subgenomic replicons. Huh7 cells were electroporated with the indicated amounts of in vitro-transcribed RNA. The mutations present in subgenomic RNAs BM4-5, BMB22-2, and BMB2-2 are listed in Table II. G418-resistant colonies were stained with crystal violet 16 days after electroporation.

(B) depicts a northern blot of total RNA extracted from cell lines FCA4 (lane 1), FCC2 (lane 2), and FCA22 (lane 3) and from pooled G418-resistant cells transfected with the indicated RNAs: BM4-5 (lane 4), BM2-2 (lane 5) and BM22-2 (lane 6). In vitro-transcribed subgenomic RNA served as a control (lane 7) for the hybridization reaction. Total RNA (5 mg) obtained from each plate was loaded onto an agarose-formaldehydegel. Genomic HCV RNA (vRNA) was detected with a riboprobe spanning the neo r gene.

FIG. 7 depicts the replication of a subgenomic replicon derived from an infectious HCV1b clone.

(A) shows colonies of Huh7 cells electroporated with RNA from HCV1bneo. Colonies were stained 16 days after the transfection.

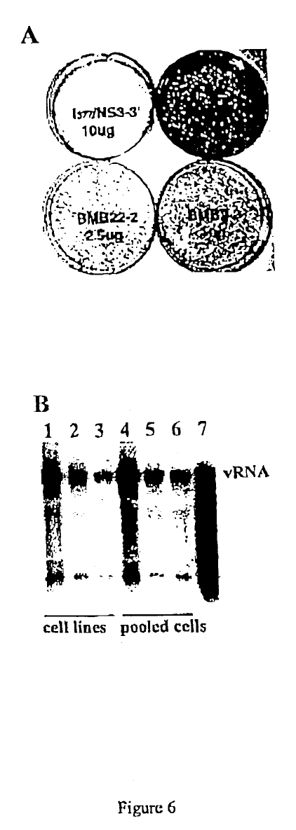

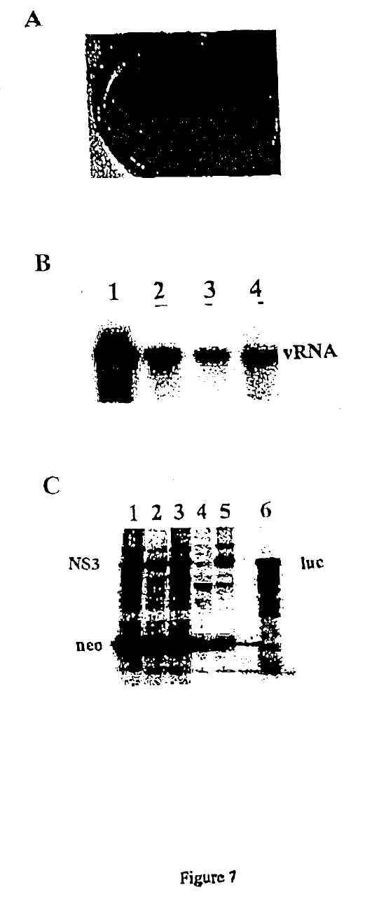

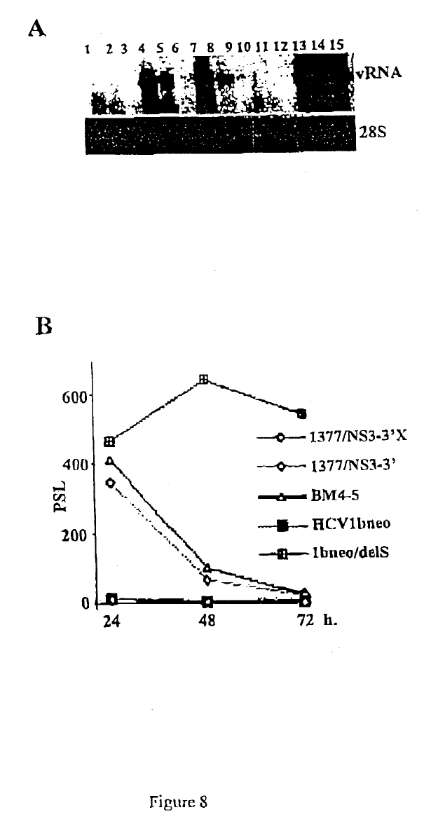

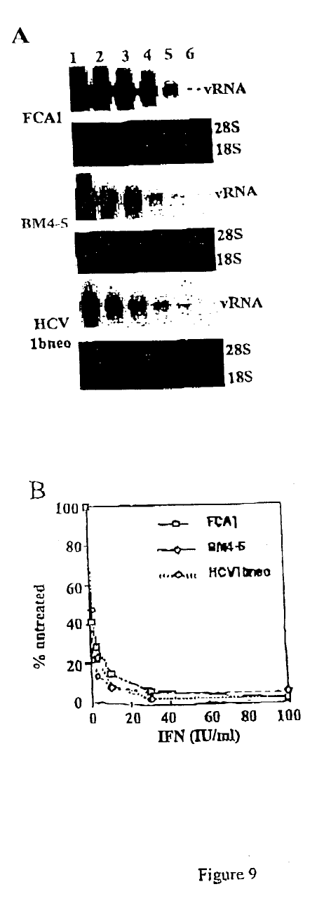

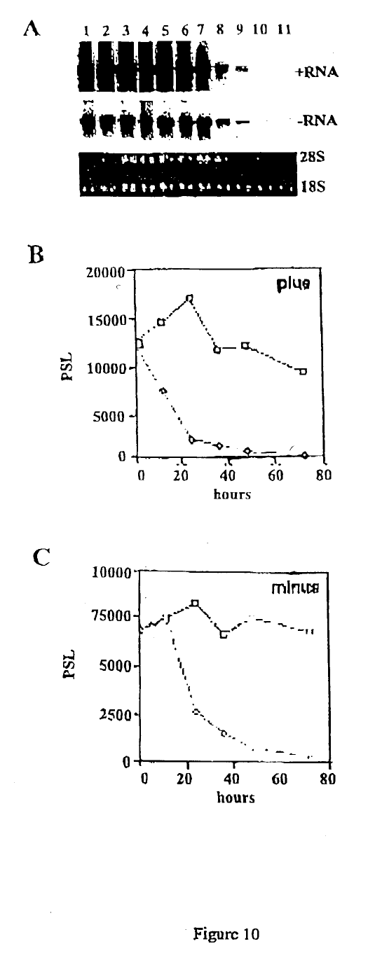

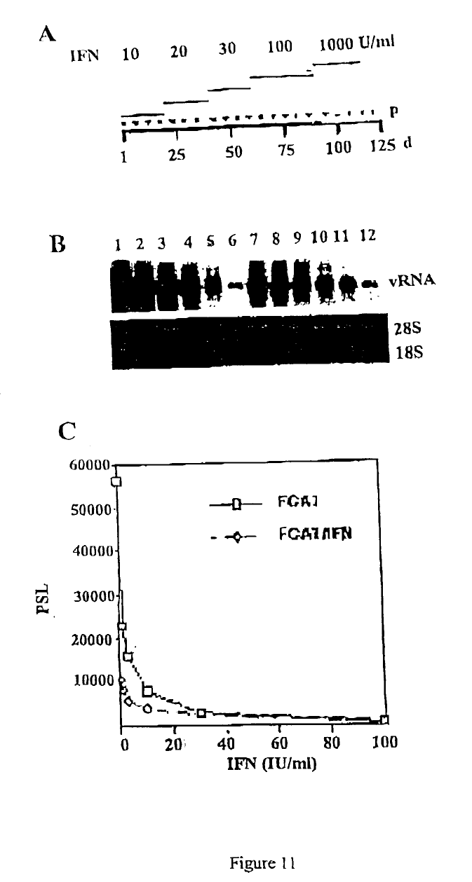

(B) depicts a northern blot of total RNA isolated from pooled cells transfected with the indicated RNAs: BM4-5 (lane 1), BM2-2 (lane 2), BM22-2 (lane 3), and HCVb1neo (lane 4). Genomic HCV RNA (vRNA) was detected as described for FIG. 6.

(C) In vitro translation of viral RNA transcribed from plasmids I377/NS3-39, HCV1bneo, HCVN-neo, D2Rneo, and D2Ineo (lanes 1 to 5). Luciferase was translated from pLuc as a positive control (lane 6). The positions of HCV NS3 and the neomycin phosphotransferase (neo) are indicated.

FIG. 8 depicts the transient replication of adapted mutants in Huh7 cells.

(A) shows a northern blot used to detect viral plus strand RNA (vRNA) present in cells transfected with I377/NS3-39X (lanes 1 to 3), I377/NS3-39 (lanes 4 to 6), BM4-5 (lanes 7 to 9), HCV1bneo (lanes 10 to 12), and 1bneo/delS (lanes 13 to 15). RNA was extracted from transfected Huh7 cells 24 (lanes 1, 4, 7, 10, and 13), 48 (lanes 2, 5, 8, 11, and 14), and 72 h (lanes 3, 6, 9, 12, and 15) after the transfection. Ethidium bromide stained rRNA (28S) served as a control for the amount of RNA loaded per lane.

(B) is a graph showing the levels of plus strand RNA in cells transfected with the indicated subgenomes 24, 48, and 72 h after the transfection as determined with a phosphorimager. PSL, arbitrary unit.

FIG. 9 illustrates the antiviral activity of IFN-alpha.

(A) shows the viral RNA (vRNA) levels present in cells treated with 0, 1, 3, 10, 30, and 100 IU of IFN-alpha/ml (lanes 1 to 6) for 72 h were determined by Northern blot analysis with a plus strand-specific RNA probe. rRNA (28S and 18S) served as a control for the amount of RNA loaded per lane. Examined were cell line FCA1 and G418-resistant pooled cells transfected with BM4-5 and HCV1bneo, respectively.

(B) shows a graph of the signal intensities that were measured with a phosphorimager and plotted as percentages of the values obtained with untreated cells (A, lane 1).

FIG. 10 shows a time course of the IFN-alpha response.

(A) depicts a Northern blot of RNA extracted from FCA1 cells incubated with 100 IU of IFN-alpha/ml (lanes 7 to 11) or without IFN-alpha (lanes 1 to 6) for 12, 24, 36, 48, and 72 h (lanes 2 to 5 and 7 to 11, respectively). Plus and minus strand RNA was detected with strand-specific RNA probes.

(B) and (C) are graphs depicting the levels of plus and minus strand RNA, respectively, as determined with a phosphorimager. PSL, arbitrary unit; diamonds, IFN-alpha-treated cells; squares, untreated cells.

FIG. 11 shows the effects of long-term treatment of FCA1 cells with IFN-alpha.

(A) shows the culturing schedule of FCA1 cells incubated for the indicated number of days (d) with increasing doses of IFN-alpha in the presence of G418 (1 mg/ml). The cells were passaged (p) at a ratio of 1:6 during the incubation with 10 to 30 IU of IFN/ml and at a ratio of 1:10 with 100 and 1,000 IU of IFN/ml at the indicated (asterisks) time points.

(B) shows a Northern blot of control FCA cells (FCA1; lanes 1 to 6) and IFN-treated cells (FCA1/IFN; lanes 7 to 12) incubated for 6 days without IFN and G418 and then with 0, 1, 3, 10, 30, or 100 IU of IFN-alpha/ml for 72 h (lanes 1 to 6 and 7 to 12, respectively). Viral RNA (vRNA) levels were determined using a plus strand-specific probe.

(C) shows the levels of viral RNA determined with a phosphorimager in FCA1 (squares) and FCA1/IFN (diamonds) cells treated with the indicated amounts of IFN-alpha. PSL, arbitrary unit.

### DETAILED DESCRIPTION OF THE INVENTION

All patent applications, patents, and literature references cited herein are hereby incorporated by reference in their entirety. Where a term is provided in the singular, the inventors also contemplate the plural of that term. The nomenclature used herein and the laboratory procedures described below are those well known and commonly employed in the art.

The present invention provides novel recombinant hepatitis C(HCV) nucleic acids containing all HCV non-translated sequences required for replication and expression, all or a portion of the HCV ORF, as well as one or more heterologous genes. In a preferred embodiment, disclosed herein are recombinant HCV-derived nucleic acids (e.g., HCVR 2 [SEQ ID NO: 2], HVCR 8 [SEQ ID NO: 3], HCVR 9 [SEQ ID NO: 4], HCVR 22 [SEQ ID NO: 5], HCVR 24 [SEQ ID NO: 6], FCA1 [SEQ ID NO: 15], FCA4 [SEQ ID NO: 16], and FCA22 [SEQ ID NO: 17]) which differ from the parental chimeric HCV replicon used for their generation (I377/NS3-3′UTR, SEQ ID NO: 1) in one or more positions located at various parts of the replicon genome (see, e.g., Table I and Table II). The positions that differ from the parental chimeric HCV replicon are identified herein according to the numbering of the nucleic acid and amino acid sequences of HCV subgenomic replicon I377/NS3-3′ (SEQ ID NO: 1; GenBank accession no. AJ242652). It is understood that the I377/NS3-3′ sequence provides a reference to identifying the amino acids or nucleotides of the HCV genome that are altered, and that the absolute number of the nucleotide or amino acid residue can be different in different replicons. Thus where a subgenomic HCV replicon of interest differs from HCV subgenomic replicon I377/NS3-3′ in, for example, the length of its overall sequence, the mutations described herein are those that correspond to the nucleotide or amino acid at the given numbered position in HCV subgenomic replicon I377/NS3-3′. Corresponding amino acid positions or nucleotide positions can be recognized by the context of the mutation, that is, the general region of an open reading frame or nucleic acid sequence region of the replicon it is found (for example, the IRES, the NS5A ORF, the 3′ untranslated region, etc.) and the nucleotide or amino acid residues that flank the mutation.

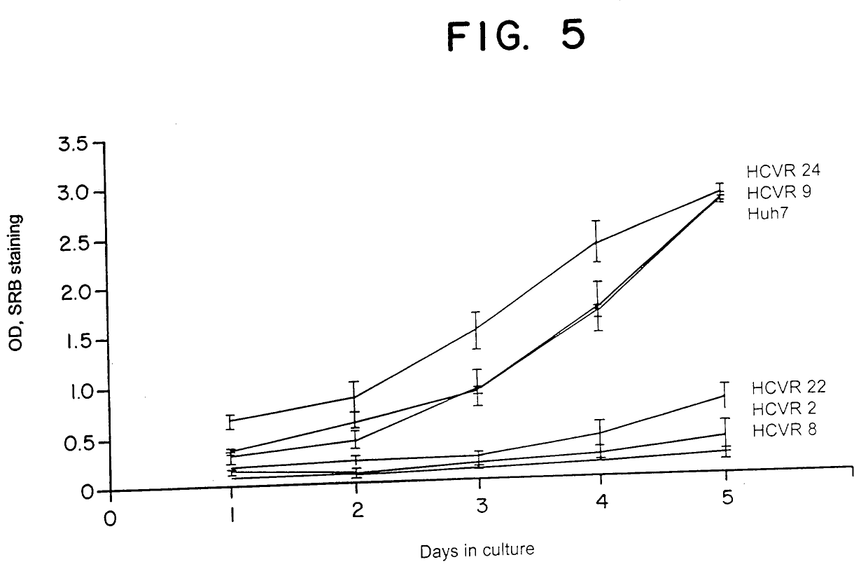

At least some of these mutant subgenomic HCV nucleic acids have a much lower cytotoxicity, as evident from their growth potential (FIG. 5) and, subsequently, have a significantly higher rate of productive transfection (Table III) compared to the previously described recombinant HCV replicons. Due to these and other advantageous properties, the subgenomic HCV nucleic acids of the present invention are capable of efficient high titer replication in the susceptible cell lines of the present invention.

In conjunction with HCV-derived nucleic acids, the present invention further provides susceptible cell lines and, in particular, novel “adapted” rapidly growing cell clones derived from human hepatoma cell line Huh-7 (e.g., clones HCVR 2, 8, 9, 22 and 24, and FCA1, 4, and 22, cell clone that are designated by the subgenomic HCV replicons they contain), said cell lines and “adapted” clones being capable of supporting efficient replication of subgenomic recombinant HCV RNAs.

Another embodiment is a sensitive high-throughput method for screening anti-HCV therapeutics by putting them in contact with the cell clones of the present invention and determining their effect on propagation of subgenomic HCV replicons.

Further provided herein is a method for generating large quantities of recombinant HCV replicons from the cell clones of the present invention. These replicons can be generated for the purpose of providing a recombinant attenuated anti-HCV vaccine and can be produced either as intracellular nucleic acids or as infectious or non-infectious recombinant viral particles.

By providing cell clones supporting efficient subgenomic HCV replication, the present invention provides (i) in vitro cell culture models of HCV propagation; (ii) systems for screening candidate anti-viral compounds and evaluating drug resistance; (iii) methods for diagnosing HCV infection, and (iv) systems for production of large quantities of HCV-derived components or recombinant viral particles for antibody generation and/or vaccine development.

### Definitions

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), in either single stranded form, or a double-stranded form. Double stranded DNA-DNA, DNA-RNA and RNA-RNA duplexes are possible. The term nucleic acid molecule, and in particular DNA or RNA molecule, refers only to the primary and secondary structure of the molecule, and does not limit it to any particular tertiary forms. In discussing the structure of particular nucleic acid molecules, sequences or regions may be described herein according to the normal convention of giving only the sequence in the 5′ to 3′ direction. A “recombinant DNA molecule” is a DNA molecule that has undergone a molecular biological manipulation.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. In the present invention, translation of HCV-derived (+) strand RNA initially yields a polyprotein, which is cleaved during post-translational processing to yield functional viral proteins.

The term “gene” means a DNA sequence that codes for or corresponds to a particular sequence of amino acids, which comprise all or part of one or more proteins or enzymes.

A coding sequence is “under the control” or “operatively associated with” transcriptional and translational control sequences in a cell when RNA polymerase transcribes the coding sequence into mRNA, which is then translated into the protein encoded by the coding sequence.

The terms “express” and “expression” mean allowing or causing the information in a gene or nucleic acid sequence to become manifest, for example producing a protein by activating the cellular functions involved in transcription and translation of a corresponding gene or nucleic acid sequence. A nucleic acid sequence is expressed in or by a cell to form an “expression product” such as a mRNA or a protein. The expression product itself, e.g. the resulting protein, may also be said to be “expressed” by the cell.

The term “polypeptide” refers to a polymer of amino acids and does not refer to a specific length of the product; thus, peptides, oligopeptides, and proteins are included within the definition of polypeptide. This term also does not refer to, or exclude, post-translational modifications of the polypeptide, for example, glycosylations, acetylations, phosphorylations, and the like.

As used herein, the term “conservative mutation” or “conservative nucleotide change” is used to define a nucleotide change, which occurs with high frequency within quasispecies. “Sequence-conservative variants” are those in which a change of one or more nucleotides in a given codon position results in no alteration in the amino acid encoded at that position. “Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrophobicity, size of the side chain, hydrogen bonding potential, and the like).

The term “hepatitis C virus” or “HCV” is used herein to define a viral species of which pathogenic strains cause hepatitis C, also known as non-A, non-B hepatitis.

As used herein, the term “viral RNA”, which includes HCV RNA, refers to RNA from the viral genome, fragments thereof, transcripts thereof, and mutant sequences derived therefrom.

As used herein, a “(+) stranded genome” or a “positive-stranded genome” of a virus is one in which the genome, whether RNA or DNA, is single-stranded and encodes a viral or virus-derived polypeptide(s). Examples of positive-stranded RNA viruses include Togaviridae, Coronaviridae, Retroviridae, Picomaviridae, and Caliciviridae. Included also, are the Flaviviridae, which were formerly classified as Togaviridae (see Fields and Knipe, Fundamental Virology, Raven Press, 1986).

As used herein, a “replicative intermediate” of an HCV genome or a “(−) stranded genome” or a “negative-stranded genome” refers to an RNA strand or fragment thereof, which is complementary to the viral genome, and which is synthesized during the course of viral replication; the replicative intermediate functions as a template for the synthesis of (+) RNA strands.

As used herein, “purified HCV virions” refers to a preparation of HCV viral or virus-like particles that have been isolated from the cellular constituents with which the virus normally associates, and from other types of viruses that may be present in the infected tissue. The techniques for isolating viruses are known to those of skill in the art, and include, for example, centrifugation and affinity chromatography.

A “construct” is a chimeric virus or nucleic acid encoding a chimeric virus, such as positive viral genomic RNA or a DNA that can be transcribed to produce viral genomic RNA.

The term “chimeric” is used herein in its usual sense: a construct or protein or virus resulting from the combination of genes from two or more different sources, in which the different parts of the chimera function together. The genes are fused, where necessary in-frame, in a single genetic construct. As used herein, the term “chimeric” refers specifically to recombinant HCV-derived nucleic acids or proteins or virions.

The term “chimeric virus genome” or “recombinant virus” or “subgenomic HCV replicon” as used herein refers to the genome of the HCV that is modified by insertion or substitution of sequences. In some instances, the virus-derived replicon which later undergoes additional changes (e.g., as a result of in vitro manipulations or in vivo selection) may be referred to as a “parent” genome or replicon. In general, according to the present invention, the recombinant virus genome will include various parts of the parent virus genome, said parts comprising without limitation genes encoding proteins involved in replication, infectivity, tropism, and life cycle.

The term “junction site” is used herein to refer to the amino acid sequence joining two different proteins of a virus-derived polyprotein that is recognized and cleaved by a protease, e.g., HCV NS3. Various HCV NS3 junction sites are known, including the NS3/NS4A, NS4A/NS4B, NS4B/NS5A, and NS5A/NS5B junction sites. Any of these can be used to substitute for an endogenous junction site of the virus used in the chimeric construct.

The term “internal ribosome entry site” or “IRES” defines a special region of some viral and cellular mRNA molecules that is capable of cap-independent binding of the ribosome to initiate translation.

As used herein, “infectious” refers to the ability of a virus to enter and replicate in a cell and to produce viral particles. Infectivity can be evaluated either by detecting virus, i.e., viral load, or by observing disease progression in an animal. Virus (viral load) can be detected by the presence of viral (+) strand RNAs and/or (−) strand replication intermediates, e.g., detected by RT-PCR or direct hybridization techniques. It can also be detected, if present in sufficient amount, by the presence of replicon-derived proteins, e.g., detected by immunoassay or biochemical techniques. In another alternative, a culture medium isolated from a cell line supporting viral replication or extracts/samples from an animal are used to infect naive cells in culture. The important aspects of the determination of viral infectivity in vivo (i.e., in infected subjects) is the development of either an acute or chronic viral infection, which, in turn, may include either overt pathology or merely replication and propagation of the virus.

The term “viral load” or “viral titer” is used herein to refer to a quantitative amount of virus in a cell culture or in an infected animal. The term “titer” can be also used to refer to a quantitative amount of virus-derived replicons produced within a susceptible cell. “Disease progression” refers to the course of disease or symptoms of an infected animal, and may include acute or chronic disease symptoms. “Pathothogenesis” is a particular indication of a disease progression, and refers to the pathogenic effects of viral infection, including morbidity and mortality. In the present invention, main pathologic effects of HCV are observed in the hepatic tissue.

An “individual” or “subject” or “animal”, as used herein, refers to vertebrates, particularly members of the mammalian species and includes, but is not limited to, rodents, rabbits, shrews, and primates, the latter including humans.

As used herein, a “biological sample” refers to a sample of tissue or fluid isolated from an individual, including but not limited to, for example, plasma, serum, spinal fluid, lymph fluid, the external sections of the skin, respiratory, intestinal and genitourinary tracts, tears, saliva, milk, blood cells, tumors, organs, and also samples of in vitro cell culture constituents (including, but not limited to, conditioned medium resulting from the growth of cultured cells, putatively viral infected cells, recombinant cells, and cell components).

As used herein, the term “quasispecies” means a collection of microvariants of a predominant HCV genome sequence (“master sequence”), said microvariants being formed in a single infected animal or even in a single cell clone as a result of high mutation rate during HCV replication.

As used herein, an “in vitro cell system” or an “extracorporeal cell system” refers to cells which are replicated outside of the body, i.e., cell systems not found in nature; as such, the term includes primary cultures and cell lines.

“Primary cultures”, as used herein, refers to a culture of cells that is directly derived from cells or tissues from an individual, as well as cells derived by passage from these cells, but not to immortalized cells.

As used herein, “cell line” refers to a population of cells capable of continuous or prolonged growth and division in vitro. The term “cell lines” also includes immortalized cells. Often, cell lines are clonal populations derived from a single progenitor cell. Such cell lines are also termed “cell clones”. It is further known in the art that spontaneous or induced changes can occur in karyotype during storage or transfer of such clonal populations. Therefore, cells derived from the cell clones referred to may not be precisely identical to the ancestral cells or cultures. According to the present invention, if such cell clones are capable of supporting efficient replication of HCV-derived RNAs without a significant decrease in their growth properties, or are demonstrated to contain subgenomic HCV RNAs with enhanced colony-forming efficiency with respect to RNA derived from subgenomic HCV construct I377/NS3-3′, they are termed “adapted” cell clones.

The term “host cell” means any cultured cell or any cell of any organism that is susceptible to infection by or propagation of a wild-type HCV or a chimeric virus construct of the invention. Host cells can further be used for screening or other assays, as described herein. A potentially susceptible cell that has not been transfected or infected with virus-derived nucleic acids is termed a “naive” cell.

The term “transfection” means the introduction of a foreign nucleic acid into a cell. The introduced gene or sequence may also be called a “cloned” or “foreign” gene or sequence, may include regulatory or control sequences, such as start, stop, promoter, signal, secretion, or other sequences used by a cellular molecular machinery. A host cell that receives and expresses introduced DNA or RNA has been “transfected” and is a “transfectant” or a “recombinant cell”. According to the present invention, if a transfection of HCV-derived nucleic acid results in its subgenomic replication and HCV-derived protein expression, the transfection is termed “productive”.

The term “antibody”, as used herein, includes both monoclonal and polyclonal antibodies. Additionally, single polypeptide chain antigen-binding proteins, see U.S. Pat. No. 4,946,778, are included within the term “antibody”.

As used herein, “epitope” refers to an antigenic determinant of a polypeptide; an epitope can comprise 3 or more amino acids in a spatial conformation unique to the epitope. Generally an epitope consists of at least 5 such amino acids and, more usually, consists of at least 8-10 such amino acids. Methods of determining the spatial conformation of amino acids are known in the art, and include, for example, x-ray crystallography and nuclear magnetic resonance (NMR).

“Immunologically identifiable with/as” refers to the presence of epitope(s) and polypeptides(s) that are also present in the designated polypeptide(s), herein usually HCV proteins. Immunological identity may be determined by antibody binding and/or competition in binding; these techniques are known to those of average skill in the art.

A polypeptide is “immunologically reactive” with an antibody when it binds to an antibody due to antibody recognition of a specific epitope contained within the polypeptide. Immunological reactivity may be determined by antibody binding, more particularly by the kinetics of antibody binding, and/or by competition in binding using as competitor(s) a known polypeptide(s) containing an epitope against which the antibody is directed. The techniques for determining whether a polypeptide is immunologically reactive with an antibody are known in the art.

As used herein, the term “immunogenic polypeptide” refers to a polypeptide that elicits a cellular and or humoral immune response, whether alone or when linked to a carrier, in the presence or absence of an adjuvant.

In a specific embodiment, the term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

### General Techniques

In accordance with the present invention there may be employed conventional molecular biology, microbiology, biochemistry, genetics and immunology techniques within the skill of the art for the production of recombinant HCV nucleic acids, HCV-replicating cell cultures, infectious viral particles, viral and recombinant proteins, antibodies, etc. Such techniques are explained fully in the literature. See, e.g., Sambrook, Fritsch and Maniatis, Molecular Cloning: A Laboratory Manual, Second Edition, 1989, Cold Spring Harbor Laboratory Press (herein “Sambrook et al., 1989”); DNA Cloning: A Practical Approach, Glover ed., Volumes I and II, 1985; Oligonucleotide Synthesis, Gait ed., 1984; Nucleic Acid Hybridization, Hames and Higgins eds., 1985; Transcription And Translation, Hames and Higgins eds., 1984; Animal Cell Culture, Freshney ed., 1986; Immobilized Cells And Enzymes, IRL Press, 1986; Perbal, A Practical Guide To Molecular Cloning, 1984; Current Protocols in Molecular Biology, Ausubel et al., eds., 1994, John Wiley & Sons.

### Construction of Recombinant HCV Replicons

In a broad aspect, the present invention is directed to genetically engineered HCV nucleic acid clones which comprise from 5′ to 3′ on the positive-sense nucleic acid (1) a functional 5′ non-translated region (NTR) comprising an extreme 5′-terminal conserved sequence, (2) at least one open reading frame (ORF) encoding a heterologous gene operatively associated with an expression control sequence, wherein the heterologous gene and expression control sequence are oriented on the positive-strand nucleic acid molecule, (3) an ORF encoding at least a portion of an HCV polyprotein whose cleavage products form functional components of HCV virus particles and RNA replication machinery, and (4) a 3′ NTR comprising an extreme 3′-terminal conserved sequence. According to thc present invention, the heterologous gene can be a drug resistance gene or a reporter gene. In a specific embodiment, where the selection marker ORF is a drug resistance gene, this gene is a neomycin resistance gene (neo) operatively associated with an internal ribosome entry site (IRES). Alternatively, the heterologous gene can be a therapeutic gene, or a gene encoding a vaccine antigen, i.e., for gene therapy or gene vaccine applications, respectively.

In a preferred embodiment, the subgenomic HCV replicons are derived from a “parent” HCV genotype 1b-based chimeric replicon I377/NS3-3′UTR (SEQ ID NO: 1, see also FIG. 3). Due to their prolonged propagation under stringent selection conditions (e.g., 1 mg/ml G418, see Example 1, infra) in susceptible cell lines, the subgenomic HCV replicons of the present invention have acquired one or more mutations.

Accordingly, also disclosed herein are positions (using the numbering of the sequence of I377/NS3-3′, SEQ ID NO: 1; GenBank accession no AJ242652) of at least some of the nucleotide and amino acid changes identified in these mutant HCV replicons (e.g., as shown in Table I and Table II), said nucleotide and amino acid changes being indicative of the genome regions which are responsible for high-titer virus replication and for virus-induced cytotoxicity. The present invention includes subgenomic HCV replicons that include at least one nucleotide or amino acid change identified using the methods of the present invention. In particular, the present invention includes any subgenomic HCV replicon comprising, from 5′ to 3′ on the positive-sense nucleic acid (1) a functional 5′ non-translated region (NTR) comprising an extreme 5′-terminal conserved sequence, (2) at least one open reading frame (ORF) encoding a heterologous gene operatively associated with an expression control sequence, wherein the heterologous gene and expression control sequence are oriented on the positive-strand nucleic acid molecule, (3) an ORF encoding at least a portion of an HCV polyprotein whose cleavage products form functional components of HCV virus particles and RNA replication machinery, and (4) a 3′ NTR comprising an extreme 3′-terminal conserved sequence, and further comprising one or more mutations identified using the methods described herein. In preferred embodiments, the present invention includes subgenomic HCV replicons that comprise one or more of the mutations disclosed in Tables I and II.

It is recognized that subgenomic HCV replicons constructs that can be used or constructed in the practice of the present invention can have one or more sequence differences with respect to I377/NS3-3′ (SEQ ID NO:1), such as, but not limited to, deletions or insertions, that can alter the number of nucleotides or amino acids with respect to HCV subgenomic replicon I377/NS3-3′. The inventors contemplate that the invention encompasses such “altered” constructs comprising mutations (such as those disclosed herein) that can be located at positions corresponding to those identified by the sequence numbering of I377/NS3-3′ (GenBank accession no. AJ242652; SEQ ID NO: 1). Corresponding amino acid positions or nucleotide positions can be recognized by those skilled in the art by the context of the mutation, that is, the general region of the replicon in which it is found (such as a particular protein-coding region or nucleotide sequence region of the replicon) and the nucleotide or amino acid residues that flank the mutation.

The actual molecular biological techniques required to generate fusion between heterologous sequences and specific fragments of HCV genome are routine. For example, a recombinant replicon can be constructed using PCR (see Lohmann et al., supra; Example 1, infra).

Naturally, as noted above, the HCV nucleic acids of the invention are selected from the group consisting of double stranded DNA, (+) strand cDNA, or (−) strand DNA, or (+) strand RNA or (−) strand RNA. Thus, where particular sequences of nucleic acids of the invention are set forth, both DNA and corresponding RNA are intended, including positive and negative strands thereof.

According to the instant invention, an HCV DNA may be inserted in a plasmid vector for transcription of the corresponding HCV RNA. Thus, the HCV DNA may comprise a promoter 5′ of the 5′-NTR on (+) strand DNA, whereby transcription of template DNA from the promoter produces replication-competent RNA. The promoter can be a member selected from the group consisting of a eukaryotic promoter, yeast promoter, plant promoter, bacterial promoter, and viral promoter. The preferred promoter is phage T7 promoter (see Example 1, infra).

In a specific embodiment, the present invention is directed to a plasmid clone, pAn/HCVR1, which can be transcribed to produce self-replicating recombinant “parental” HCV RNA transcript I377/NS3-3′UTR (see FIG. 3).

Also disclosed herein are cRNA molecules transcribed from the recombinant HCV-derived DNA plasmid clones set forth above. These cRNA molecules are used for transfection into the cell lines of the present invention.

The present invention further advantageously provides DNA plasmid vectors containing inserts derived from the mutant RNA replicons disclosed herein using reverse transcription followed by PCR-amplification or other molecular biology techniques well known in the art.

Naturally, the invention also includes derivatives of all of the disclosed nucleic acids, selected from the group consisting of derivatives produced by substitution of homologous regions from other HCV isolates or genotypes; derivatives produced by mutagenesis; derivatives selected from the group consisting of infectious, adapted, live-attenuated, replication competent non-infectious, and defective variants; derivatives comprising additional heterologous gene(s) operatively associated with an expression control sequence; and derivatives consisting of a functional fragment of any of the above-mentioned derivatives. Alternatively, portions of the disclosed nucleic acids, such as the 5′ NTRs, the polyprotein coding regions, the 3′-NTRs or more generally any coding or non-translated region of the HCV genome, can be substituted with a corresponding region from a different HCV genotype to generate a new chimeric clone, or by extension, clones of other isolates and genotypes. For example, an HCV-1a or -2a polyprotein coding region (or consensus polyprotein coding regions) can be substituted for the HCV-1b polyprotein coding region of the deposited clones.

By providing the engineered HCV nucleic acids of the invention, the present inventors have made it possible to dissect the HCV replication machinery and protein activity. The instant invention has also made possible the preparation of various HCV derivatives such as attenuated, expressing heterologous gene(s), replication-competent non-infectious, replication-defective infection-competent, and replication-defective non-infectious. The recombinant HCV DNA clones or RNAs of the invention can also be used in numerous methods, or to derive authentic HCV components, as set forth below.

### Production of “Adapted” Cell Clones Supporting Efficient Subgenomic HCV Replication

The instant invention further provides a method for propagating the disclosed recombinant HCV-derived nucleic acids in vitro comprising culturing a cell line transfected or infected with an appropriate amount of HCV RNA, e.g., as produced from the plasmid clones recited above, under conditions that permit replication of said HCV-derived RNA.

By providing novel recombinant HCV nucleic acids, in particular, nucleic acids containing mutations in the regions responsible for cytotoxicity and/or replicon titer, the present invention provides a method for generating stable cell clones supporting efficient replication of subgenomic HCV RNAs.

Accordingly, in conjunction with the recombinant nucleic acids disclosed herein, the present invention advantageously provides susceptible stable cell lines which, upon transfection or infection, can support high titer replication of the disclosed HCV-derived nucleic acids. The susceptible cell lines of the invention include without limitation human hepatoma cell lines Huh-7, HepG2, and PH5CH; T. belangeri liver cell line MBTL; human diploid fibroblast cell line VERO; secondary monkey kidney cell line CV-1; T cell lines MT-2, HPBMa10-2, and MOLT-4, and B cell line Daudi.

As disclosed herein, the HCV-replicating stable cell lines of the present invention can be characterized by the growth rates which are not less than 10% of the growth rate of the corresponding naïve (non-transfected) cell lines. In a preferred embodiment, these growth rates are not less than 25% of the growth rate of the corresponding naïve (non-transfected) cell lines, or, even more preferably, not less than 90% of the growth rate of the corresponding naïve (non-transfected) cell lines. Alternatively or in addition, the HCV-replicating stable cell lines of the present invention can be characterized by having subgenomic HCV replicons that have a greater efficiency of colony formation than subgenomic HCV replicon I377/NS3-3′, when transfected into a susceptible cell line.

In a preferred embodiment, the susceptible cell line is the human hepatoma cell line Huh-7. As disclosed herein, the “adapted” cell clones derived from Huh-7 cell line support efficient replication of subgenomic HCV nucleic acids of the present invention and can be characterized by the growth properties which are similar to or indistinguishable from the naive (i.e., non-transfected) Huh-7 cells (see, e.g., FIG. 5). Besides promoting the generation of novel quasispecies of recombinant replicons (e.g., containing mutations leading to lowered cytotoxicity and increased replication efficiency) these “adapted” cell clones might have acquired certain mutations in their own genomes, said mutations distinguishing them from the parental naive cells and leading to their improved ability to support replication of subgenomic HCV RNAs. Some of the most efficiently propagating cell clones of the present invention, i.e., HCVR 2, 8, 9, 22 and 24, have been deposited with the American Type Culture Collection (ATCC), 10801 University Boulevard, Manassas, Va. 20110-2209, USA on Sep. 20, 2000, and assigned Accession Nos. PTA-2489, PTA-2490, PTA-2486, PTA-2487, and PTA-2488, respectively. Other cell clones of the present invention, i.e., FCA1, FCA4, and FCA22, were deposited with the American Type Culture Collection (ATCC), 10801 University Boulevard, Manassas, Va. 20110-2209, USA on ______ [PLEASE PROVIDE DATE OF DEPOSIT], and assigned Accession Nos. ______ [PLEASE PROVIDE ATCC ACCESSION NOS.], respectively.

The methods for detection of subgenomic HCV replicons in the cell lines of the present invention are well known in the art and include without limitation (i) Nothern blot or strand-specific reverse transcriptase-polymerase chain reaction (RT-PCR) detection of (−) strand replicative RNA intermediates; (ii) determination of an increase of (+) strand RNA using either b-DNA assay or quantitative RT-PCR, (iii) detection of expression of a heterologous reporter gene encoded by a recombinant HCV replicon (e.g., by assaying fluorescence, luminescence or enzymatic activity) and (iv) detection of HCV-derived proteins (e.g., by Westen blot or immuno-fluorescence).

The permissive cell lines that are identified using the nucleic acids of the invention are very useful, inter alia, for studying the HCV infection and propagation, isolating functional components of HCV, and for sensitive, fast diagnostic and therapeutic applications.

In a specific embodiment, the permissive cell line and the “adapted” cell clones of the present invention are used to select novel mutant recombinant HCV replicon quasispecies. By providing HCV nucleic acids comprising a selectable marker, the present invention provides a method for selecting novel mutated chimeric HCV replicons characterized by low cytotoxicity, high productive transformation efficiency, and high replicon titer attained; said method comprising (i) transformation of a susceptible cell line followed by (ii) prolonged culturing under stringent selection conditions which allow only the survival of cells supporting high titer HCV replication, and (iii) isolation of recombinant replicons from the cell clones characterized by the best growth properties. Methods for identification of the novel mutations contained within these selected replicons are well known in the art and include, for example, a sequence of steps: reverse transcription->PCR amplification->automated sequencing->computer sequence analysis.

### Production of Recombinant Virions

The invention further provides a method for producing recombinant HCV virus-like particles, comprising isolating HCV virus-like particles from the cell lines of the invention (or their culture medium) under conditions that permit subgenomic HCV replication and virus particle formation. The present invention extends to a recombinant HCV-derived virus particle comprising a replication-competent subgenomic HCV RNA, or a replication-defective HCV-derived RNA; said recombinant HCV virus particle being either infection-competent or infection-defective. In a specific embodiment the recombinant viral particles produced in the cell lines of the present invention provide an attenuated recombinant vaccine to be administered to an individual to produce an anti-viral immune response. Alternatively, isolated HCV-derived proteins expressed in the cell lines of the present invention can represent starting materials for an HCV vaccine. Preferably, a vaccine of the invention includes a pharmaceutically acceptable adjuvant. Representative but not limiting examples of adjuvants include Complete and Incomplete Freund's Adjuvant, detoxified endotoxins, mineral oils, surface active substances such as lipolecithin, pluronic polyols, polyanions, peptides, oil or hydrocarbon emulsions, immunostimulatory compounds, Bacille Calmette-Guerin (BCG), and the synthetic adjuvant QS-21 (McCune et al., Cancer, 1979; 43:1619).

### Diagnostic and Therapeutic Applications

By providing for insertion of heterologous genes in the recombinant HCV nucleic acids, the present invention provides a method for transducing an animal susceptible to HCV infection with said heterologous genes by administering an amount of the HCV RNA to the animal either directly or in the form of infectious recombinant viral particles produced in the cell lines disclosed herein. HCV-mediated introduction of heterologous genes in susceptible animals can be useful, for example, for gene therapy or gene vaccination which is targeted to hepatic tissues.

Also provided is an in vitro cell-free assay system for HCV replication comprising HCV-derived template cRNA of the invention, e.g., as transcribed from a plasmid of the invention as set forth above, functional HCV replicase components, and an isotonic buffered medium comprising ribonucleotide triphosphate bases. These elements provide the replication machinery and raw materials (NTPs).

In a further embodiment, the invention provides a method for producing novel polyclonal antibodies to HCV-derived proteins and/or recombinant viral particles comprising administering an immunogenic amount of HCV-derived proteins isolated from the cell cultures or the in vitro cell-free system described above to an animal, and isolating generated anti-HCV antibodies. A further method for producing antibodies to HCV comprises screening a human antibody library for reactivity with HCV-derived proteins of the invention and selecting a clone from the library that expresses a reactive antibody. Alternatively, novel monoclonal anti-HCV antibodies can be produced in hybridoma cell lines using techniques well known in the art.

According to the present invention, the usefulness of newly generated antibodies can be assayed by measuring their affinity and specificity (e.g., upon their application to isolated HCV-derived antigens and/or HCV replicating cell lines of the invention and/or liver tissue sections from chronically infected animals).

The novel anti-HCV antibodies disclosed herein may be used diagnostically, e.g., to detect the presence and/or propagation of HCV in a cell culture or in an animal. Alternatively, these antibodies may be used therapeutically, e.g., in passive immunotherapy.

The present invention further advantageously provides methods for screening (both in vitro and in vivo) for agents capable of modulating HCV infection and/or replication and/or virion assembly. Such methods include administering a candidate agent to HCV infected or transfected cell line(s) of the invention, and testing for an increase or decrease in a level of subgenomic HCV replication or HCV-associated protein expression compared to a level of HCV replication or HCV-associated protein expression in a control cell line transfected with replication-defective construct or in the same cell line prior to administration of the candidate agent, wherein a decrease in the level of HCV replication (i.e., decrease in intra- or extracellular levels of recombinant HCV (+) and/or (−) RNA) or HCV-associated protein expression (e.g., decrease in intra- or extracellular levels of a reporter protein) is indicative of the inhibitory activity of the agent. Agent-mediated inhibition of virion formation can be detected microscopically (performed directly or after immunostaining); and changes in infectivity of generated HCV virus particles can be assayed by isolating them from the cell culture medium and applying to naive cells or a susceptible animal model.

In a specific embodiment, HCV-replicating cell lines of the present invention provide a convenient system for high-throughput initial screening of potential anti-HCV therapeutics. Such high-throughput screening system involves applying test compounds to the microcultures of cell clones supporting subgenomic HCV replication (growing, e.g., in 96- or 324-well microtiter plates) followed by measuring changes (e.g., using multi-plate readers or scanners) in HCV replication and/or HCV-associated protein expression and/or HCV infectivity. According to the instant invention, candidate therapeutic compounds include without limitation small molecule enzyme inhibitors (e.g., helating agents), inhibitory peptides, inhibitory (e.g., transdominant-negative) proteins, antibodies, ribozymes, and antisense nucleic acids.

A further method for screening for agents capable of modulating HCV propagation involves a cell-free system described above. This method (which can be also performed in a high-throughput format) comprises contacting the in vitro cell-free system of the invention with a candidate agent and testing for an increase or decrease in a level of HCV replication or HCV-associated protein expression compared to a level of HCV replication or HCV-associated protein expression in a control cell-based system or a control cell-free system prior to administration of the candidate agent; wherein a decrease in the level of HCV replication or HCV-associated protein expression compared to the control level is indicative of the ability of the agent to inhibit HCV propagation.

As disclosed herein, the anti-HCV therapeutic compounds identified using the initial in vitro screening methods of the present invention can be further characterized for their ability to affect subgenomic HCV propagation using secondary screens in cell cultures and/or susceptible animal models. Based on the tropism of the HCV, a preferred small animal model of the present invention is a tree shrew Tupaia belangeri chinensis. A preferred large animal model is a chimpanzee. Test animals will be treated with the candidate compounds that produced the strongest inhibitory effects in cell culture-based assays (control animals would not be treated, and, if available, a positive control could also be employed). A compound that protects animals from infection by the chimeric virus and/or inhibits viral propagation leading to pathogenicity, would be an attractive candidate for development of an agent for treatment/prevention of HCV infection. In addition, the animal models provide a platform for pharmacokinetic and toxicology studies.

The present invention also has significant diagnostic implications. For example, HCV-specific antibodies prepared according to the invention can be used to detect HCV presence and/or propagation in various biological samples. On the other hand, the invention provides an in vitro method for detecting antibodies to HCV in a biological sample from a subject comprising contacting said sample with HCV-replicating cells, cellular fractions, isolated HCV-derived proteins, or HCV-derived viral particles prepared as described above, under conditions that permit interaction of HCV-specific antibodies in the sample with the HCV protein(s), followed by detecting binding of the antibodies in the sample to the HCV-derived protein(s), wherein said binding is indicative of the presence of HCV infection in the subject from which the sample was derived. In the foregoing methods, the biological sample can be derived without limitation from blood, serum, plasma, blood cells, lymphocytes, or liver tissue biopsy. Techniques for isolating proteins and cellular fractions useful in the foregoing diagnostic methods are also well known in the art.

In a related aspect, the invention also provides a test kit for HCV diagnostics comprising anti-HCV antibodies, HCV virus components and/or cell lines permissive for HCV replication and expressing these components.

Taken together, the primary object of the present invention is to provide nucleic acids encoding recombinant HCV-derived replicons, which are capable of efficient propagation in a permissive cell culture system.

A related object of the invention is to provide susceptible cell lines (and “adapted” cell clones derived from them) which support high titer replication of recombinant HCV-derived nucleic acids.

By providing cell clones supporting efficient subgenomic HCV replication, the present invention provides (i) in vitro cell culture models of HCV propagation; (ii) systems for screening candidate anti-viral compounds and evaluating drug resistance; (iii) methods for diagnosing HCV infection, and (iv) systems for production of large quantities of HCV-derived components or recombinant viral particles for antibody generation and/or vaccine development.

### EXAMPLE 1

### Generation of Stable Rapidly Growing Huh-7 Hepatoma Cell Lines Which Support Efficient Replication of Subgenomic Hepatitis C Virus RNAs

### Materials and Methods

Generation of subgenomic HCV RNAs. Parental recombinant HCV DNA construct for expression of subgenomic HCV RNAs was produced by Operon Technologies, Inc. (Alameda, Calif.) using the sequence described by Lohmann et al. (Science, 1999, supra).

Bicistronic HCV-derived DNA fragment I377/NS3-3′UTR (SEQ ID NO: 1, FIG. 3) was chemically synthesized and inserted into a modified pUC19 vector under the control of T7 promoter to produce expression plasmid pAn/HCVR1. Plasmid DNA was linearized with Sca I and used for in vitro transcription reactions with Megascript T7 In Vitro Transcription Kit (Ambion) according to manufacturer's instructions. After in vitro transcription and DNase treatment (to remove template DNA), RNA was extracted with acid phenol (Kedzierski and Porter, BioTechniques, 1991, 10:210) and used for transfections.

Cell line growth. Human hepatoma cell line Huh-7 (Nakabayashi et al., Cancer Res., 1982, 42:3858-3863) was grown in Dulbecco's Modified Eagles Medium (DMEM), supplemented with 10% Fetal Bovine Serum (FBS), Penicillin/Streptomycin, 2 mM glutamine, and non-essential amino acids, in a humidified atmosphere containing 5% CO₂. Cells were passaged 1-2 times per week at a split ratio of 1:4-1:5, using trypsin/EDTA solution (0.05%/0.02%). All tissue culture reagents were from Gibco.

Cell transfection and selection of efficiently growing clones supporting high titer subgenomic HCV replication. RNA (10-30 μg) was electroporated into 0.8×10⁷ cells using Gene Pulse instrument (Bio-Rad, 0.4 cm-cuvette, 450V, 960 Fa). Transfection of a construct directing the expression of firefly luciferase was used to optimize transfection efficiency. Following electroporation, cells were seeded on 10-cm culture dishes in 10 ml of growth medium. After 48 hours, G418 was added at 1 mg/ml, and the medium was changed every second day until (after 3 weeks of selection) resistant colonies were transferred to 96-well plates and passaged 1-2 times a week.

The fraction of HCV-positive cells in the total population was monitored by immunofluorescence using polyclonal antibodies raised against non-structural HCV proteins (see below) and by RT-PCR (see below; see also Lanford et al., Virology, 1994, 202:606; Shimizu et al., Proc. Natl. Acad. Sci. USA, 1992, 89:5477; Mizutani et al., J. Virol., 1996, 70:7219; Ikeda et al., Virus Res., 1998, 56:157; Fournier et al., J. Gen. Virol., 1998, 79:2376).

Stable cell clones were expanded and culture conditions were optimized to promote rapid cell growth and HCV RNA replication. Growth rates of the clones were monitored by sulphorhodamine B staining for total cellular protein. The clones which (i) had the growth properties better or indistinguishable from the naive Huh-7 cells (see, e.g., FIG. 5) and (ii) supported efficient subgenomic HCV RNA replication (as determined by quantitative RT-PCR) were chosen for further analysis.

RT-PCR assays. Total RNA was prepared from transfected cells, and serial dilutions were used for RT-PCR amplification of (i) NSSB region between nt 7435 and 7750 using primers GCCCTAGATTGTCAGATCTACG and ATAAATCCAACTGGGACGCAGC (SEQ ID NOS: 7 and 8, respectively), (ii) NS5B region between nt 7360 and 7800 using primers CCTTGTGGGCAAGGATGATCC and GACAGGCTGTGATATATGTCTCC (SEQ ID NOS: 9 and 10, respectively), and (iii) neo region between nt 650 and 1110 using primers GTTCTTTTTGTCAAGACCGACC and CCACCATGATATTCGGCAAGC (SEQ ID NOS: 11 and 12, respectively). As a control, RT-PCR was performed without template DNA or using total RNA isolated from naive Huh-7 cells.

Antibodies. Rabbit polyclonal antisera specific for HCV NS3 or NS5A (Lohmann et al., Science 1999, 285:110-113) were obtained from Ralf Bartenschlager (Institute for Virology, Johannes-Gutenberg University, Mainz, Germany). A mouse monoclonal antibody to HCV NS4B was from Virogen, Inc. (Watertown, Mass.).

In situ detection of HCV antigens using immunofluorescence. Monolayer cultures on glass coverslips were fixed with methanol-acetone mixture (1:1) for 2 min, and dried for 10 min at room temperature. Alternatively, cells were fixed directly in 6-well plates (Costar). Dried cell monolayers were blocked with 2% bovine serum albumin (BSA) in phosphate-buffered saline (PBS) for 10 min at room temperature. Double staining of cells was achieved by using rabbit anti-HCV primary antibody followed by the addition of 2 mg/ml of 4′,6-diamidino-2-phenylindole (DAPI, Sigma) to stain the cellular DNA, and then secondary antibody (goat anti-rabbit IgG polyclonal antibody conjugated with rhodamine; Sigma). All primary and secondary antibodies were diluted 1:100 in blocking solution. After mounting, the samples were viewed with an Olympus BX60 microscope with a ×20 or ×40 objective and specific filter blocks. Images were acquired using a charge-coupled device camera (Olympus, model FKH025144) and processed using Adobe Photoshop and Canvas software.

Detection of HCV antigens by immunoblotting. Monolayer cultures of cells growing on 6-well plates were lyzed and subjected to electrophoresis in 12% or 4-20% gradient SDS-polyacrylamide gels (Laemmli, Nature, 1970, 227:680-685). After electrotransfer of proteins to a nitrocellulose filter, HCV antigens were detected using rabbit anti-HCV antibodies and Enhanced Chemiluminescence (ECL) detection kit (Amersham).

Analysis of subgenomic HCV replicons isolated from rapidly growing Huh-7 cell clones. Total RNA was isolated from HCVR 2 (SEQ ID NO: 2), HVCR 8 (SEQ ID NO: 3), HCVR 9 (SEQ ID NO: 4), HCVR 22 (SEQ ID NO: 5) and HCVR 24 (SEQ ID NO: 6) rapidly growing Huh-7 cell clones carrying replicating subgenomic HCV constructs. The whole replicon RNA was reverse-transcribed using oligonucleotide primers CTCGTATGTTGTGTGGAA and GTCGCTCTCGAGGCACATA (SEQ ID NOS: 13 and 14, respectively) designed and synthesized by Operon Technologies (Alameda, Calif.) and PCR-amplified in overlapping 0.7-1.2 kb-fragments. Total PCR products were sequenced rather then individual cDNA clones, in order to identify predominant mutations. Typically, every region was sequenced in both directions, using at least two PCR products and several primers for each direction. Sequencing was performed on a contract basis at the Children's Hospital of Philadelphia (Philadelphia, Pa.). The resulting sequences were analyzed using Sequencher™ 3.1 sequence alignment software.

Results and Discussion Despite the availability of cloned infectious genomes, molecular studies of HCV replication and the development of antiviral drugs have been hampered by the low efficiencies of currently available cell culture systems (see overview in the Background Section).

To overcome these limitations, an efficient cell culture system was established that is based on the transfection of recombinant HCV-derived RNAs. Using chemical DNA synthesis, pAn/HCVR1 DNA plasmid was constructed, said plasmid encoding a selectable bicistronic replicon (I377/NS3-3′UTR; SEQ ID NO: 1) containing HCV 5′ NTR (HCV IRES), the neomycin phosphotransferase (neo) gene, the IRES of the encephalomyocarditis virus which directs translation of HCV sequences from NS3 up to NS5B, and HCV 3′ NTR (see Materials and Methods and FIG. 3).

A 5′-flanking T7 RNA polymerase promoter and an engineered restriction site at the 3′ end of pAn/HCVR1 allowed for in vitro production of run-off RNA transcripts corresponding to a selectable bicistronic replicon. These in vitro transcripts (termed “parental” replicons) were transfected into Huh-7 human hepatoma cells. Particular care was taken to remove template DNA, which might otherwise integrate into transfected cells and confer G418 resistance independent of HCV replication (see Materials and Methods). The efficiency of transfection was optimized using luciferase mRNA.

In two independent transfection experiments, after 3 weeks of G418 selection (at 1 mg/ml), a total of 33 drug-resistant clones were obtained. These clones were transferred to 96-well plates and passaged 1-2 times a week. Seven cell clones were successfully expanded and were selected for further study.

At five weeks posttransfection clones were analyzed by RT-PCR for the presence of HCV RNA (NS5B region was amplified, see Materials and Methods). Clones HCVR 2, 8, 9, 22 and 24 produced PCR fragments of the expected size (FIG. 4). These results were confirmed by repeating RT-PCR with three other primer pairs: two corresponding to NS5B, and one to neo gene.

Although the positive RT-PCR data and the presence of G418 resistance 5-7 weeks posttransfection are strong indications of subgenomic HCV replication, there is still a possibility that both of these effects are due to traces of the input replicon RNA. To rule out this possibility, selected Huh-7 clones which produced positive results by RT-PCR were assayed for the expression of HCV-derived NS proteins. Thus, selected clones HCVR 2, 8, 9, 22 and 24 were analyzed by immunoflurescence microscopy using anti-NS3, NS4, and NS5A antibodies (see Materials and Methods). All three viral proteins were detected in all five cell clones. Similarly to wild-type HCV which replicates in the cytoplasm, the antigens derived from the recombinant replicons of the present invention also localized to the cytoplasm. NS5A protein was expressed in a substantial fraction of cells (at least 30%) in clones HCVR 2 and HCVR 8, but not in naive Huh-7 cells. Similar pattern was observed for NS3, NS4 and NS5A proteins in all five selected cell clones.

The presence of HCV replicon-derived antigens in clones HCVR 2, 8 and 9 was also analyzed by Western blotting, and both NS5A and NS3 proteins were detected. Interestingly, when the immunofluorescence detection of NS5A was repeated at 6, 7, 8, 9, and 10 weeks posttransfection, it was found that the fraction of positive cells gradually increased with time, and at 10 weeks posttransfection over 90% of cells (in clones HCVR 8, 9, 22, 24) became HCV-positive.

Importantly, dramatic changes in cell growth potential were observed over time in several isolated cell clones, which were efficiently replicating subgenomic HCV. Initially (similarly to observations of Lohmann et al., Science, 1999, supra), all HCV-replicating cell lines were growing much slower as compared to the parental naive cell line, Huh-7 (i.e., growth rate of the cell lines was not more than 1% of the growth rate of naive Huh-7 cells). At certain points in time, however, (starting 6 weeks posttransfection) the growth of some of the clones began to accelerate (10-fold or more), ultimately reaching the growth rates of parental Huh-7 cells. As shown in FIG. 5, clones HCVR 9 and 24 grew at the same rate as the parental (non-transfected) Huh-7 cells. Similarly, clones HCVR 2, 8 and 22 grew only 4-fold slower than non-transfected Huh-7 cells. This is in contrast to the growth rates of cells initially prepared by reproducing experiments of Lohmann et al., which had a reduction of growth rate of at least 99% as compared to the naive cell line. Such increase in growth rates of selected clones was not previously reported and coincided with the emergence of detectable HCV-derived antigens and HCV RNA, suggesting that the observed changes were not associated with the loss of HCV replication.

In the studies of recombinant HCV replicons propagating in Huh-7 cells, Lohmann et al. did not observe any time-dependent increase in the efficiency of productive transfection and have not obtained host cell clones with normal growth properties. On the basis of these studies Lohmann et al. have hypothesized that HCV RNA replication and/or the expression of HCV NS proteins is always disadvantageous to cells. Moreover, Lohmann at el. failed to find any mutations in the 10 replicons that they have sequenced and concluded that formation of an “adapted” replicon would be rare and that the inefficiency of their system is more likely due to particular host cell conditions or factors present in only a few cells.

In contrast, the data reported here support a hypothesis that at least some of the selected replicons are “attenuated”, i.e., contain mutations that are responsible for their lowered cytotoxicity and high frequency of productive transfection. To identify these mutations, replicon RNAs isolated from all five selected cell clones (HCVR 2, 8, 9, 22 and 24) were amplified using RT-PCR and sequenced. The obtained sequences (SEQ ID NOS: 2-6) were analyzed for the presence of mutations by comparing them to the sequence of the parental I377/NS3-3′UTR clone (SEQ ID NO: 1). As shown in Table I, mutations are located in various parts of the replicon genome. Further analysis of these mutations may provide important information about the evolution of the virus and the viral genes responsible for replication efficiency and cytotoxicity.

TABLE I

Mutations in Huh-7 replicon clones

| | | | | | |
|---|---|---|---|---|---|
| Clone# | position | change (nt) | change (aa code) | change aa | Gene |

| | | | | | |
|---|---|---|---|---|---|
| HCVR2 | 1234 | C to A | | | EMCV IRES |
| HCVR2 | 2527 | A to G | ATG to GTG | M to V | NS3 |
| HCVR2 | 5288 | G to T | CGT to CTT | R to L | NS5A |
| HCVR8 | 2330 | A to G | GAA to GGA | E to G | NS3 |
| HCVR8 | after 4847 | AAA insertion | added K | NS5A | |
| HCVR8 | 5366 | C to T | GCA to GTA | A to V | NS5A |
| HCVR9 | 1236 | | | | EMCV IRES |
| HCVR9 | 1905 | A to G | CAA to CAG | Silent | NS3 |
| HCVR9 | 3797 | A to G | AAG to AGG | K to R | NS4A |
| HCVR9 | 4364 | T to C | GTT to GCT | V to A | NS4B |
| HCVR9 | 4848 | C to A | AAC to AAA | N to K | NS5A |
| HCVR22 | 2330 | A to G | GAA to GGA | E to G | NS3 |
| HCVR22 | 3935 | A to G | CAA to CGA | Q to R | NS4B |
| HCVR22 | 5320 | G to A | GCC to ACC | A to T | NS5A |
| HCVR24 | 1760 | C to T | | | EMCV IRES |
| UCVR24 | 3016 | G to T | GAC to TAC | D to Y | NS3 |
| HCVR24 | 5336 | G to T | AGC to ATC | S to I | NS5A |

In summary, in contrast to all previous reports, using prolonged selection in the presence of G418, the present inventors have generated HCV-replicating “adapted” cell clones, which grow at the rate, which is similar to or indistinguishable from the non-transfected parental Huh-7 cell line. These cell clones support efficient propagation of recombinant HCV replicon mutants at least some of which have unusually low cytotoxicity leading to high replication titers and high cell growth rate.

Cell clones HCVR 2, 8, 9, 22 and 24, were deposited with the American Type Culture Collection (ATCC), 10801 University Boulevard, Manassas, Va. 20110-2209, USA on Sep. 20, 2000, and assigned Accession Nos. as set forth below:

ATCC Deposits

| HCV cell clone | Accession No. |
|---|---|
| HCVR 2 | PTA-2489 |
| HCVR 8 | PTA-2490 |
| HCVR 9 | PTA-2486 |
| HCVR 22 | PTA-2487 |
| HCVR 24 | PTA-2488 |

### EXAMPLE 2

### Generation of Stable Huh-7 Hepatoma Cell Lines Which Support Efficient Replication of Subgenomic Hepatitis C Virus RNAs

### Materials and Methods

Cell Culture. Huh-7 clones were grown in Dlubecco's modified Eagle's medium (DMEM) (Gibco-BRL) supplemented with 10% fetal bovine serum, L-glutamine, nonessential amino acids, penicillin, and streptomycin.

Construction of recombinant plasmids. HCV genotype 1b replicon I377/NS3-3′ (GenBank accession no. AJ242652; Lohman et al. (1999) Science 285: 110-113) was assembled and cloned from chemically synthesized DNA oligomers. HCV1bneo was engineered by replacing a BsrGI-ScaI fragment in I377/NS3-3′ with a BsrGI-XbaI fragment from plasmid HCV-N (Beard, et al. (1999) Hepatology 30: 316-324). BM4-5 was constructed by replacing an EcoRI (position 5083)-XhoI (position 5570) fragment of I377/NS3-3′ with the corresponding fragment, which was cloned by reverse transcription PCR (RT-PCR) with RNA isolated from cell line FCA4. BMB2-2 and BM22-2 were made by replacing an SspI (position 4045)-EcoRI (position 5083) fragment in I377/NS3-3′ with the corresponding fragment, which was cloned by RT-PCR with RNA isolated from cell lines FCC2 and FCA22, respectively. Plasmid 1bneo/delS was derived from

HCV1bneo by deletion of nucleotides 6955 to 6957 (HCV-N; accession no. AF139594) using oligomer-directed site-specific mutagenesis. I377/NS3-3′X was constructed by filling in the XhoI site in I377/NS3-3′ (position 5570) with Klenow DNA polymerase. The nucleotide sequences of the substituted fragments were determined with an ABI sequencer.

In vitro transcription and purification of RNA. Plasmids were linearized with ScaI (I377/NS3-3′ and its derivatives), XbaI (HCV1bneo), or MluI (D2Rneo) and transcribed in vitro with the MEGAscript kit (Ambion, Austin, Tex.) in accordance with the manufacturer's protocol. For a 20-microliter reaction mixture, transcription was stopped by the addition of 4 U of DNase I and the mixture was incubated for 45 min at 37° C. RNA was extracted with 1 ml of TRIzol reagent (Gibco-BRL) and precipitated with 500 microliters of isopropanol. RNA pellets were washed once with 75% ethanol and dissolved in RNase-free water. To remove the residual amount of template DNA, RNA preparations were extracted once with acid phenol, precipitated with ethanol, and resuspended in RNase-free water as described by Kedzierski and Porter (1991) BioTechniques 10: 210-214.

RNA transfection. Subconfluent Huh7 cells were trypsinized and washed once with complete DMEM and once with serum-free DMEM-F12 medium. Cell pellets were resuspended in serum-free DMEM-F12 medium at a density of 10⁷ cells/ml. To 200 microliters of the cell suspensions in an electroporation cuvette (0.2-cm gap; BTX, San Diego, Calif.) 1 to 10 micrograms of in vitro-transcribed RNA was added. The cells were immediately electroporated with an ECM 630 apparatus (BTX) set to 200 V and 1,000 microF. After electroporation the cell suspension was kept for 5 min at room temperature and then diluted into DMEM supplemented with 10% fetal bovine serum and nonessential amino acids and seeded into a 10-cm-diameter petri dish. After 24 h, G418 was added to obtain a final concentration of 1 mg/ml, and medium was changed every other day. G418-resistant colonies became visible after 2 to 3 weeks.

Northern blot hybridization. Total cellular RNA was extracted with TRIzol reagent (Gibco-BRL). Five to 10 micrograms of total RNA was electrophoresed through a 1.0% agarose gel containing 2.2 M formaldehyde and transferred to a nylon membrane and immobilized by UV cross-linking (Stratagene). Hybridization was carried out with [³²P]UTP-labeled in vitro-transcribed RNA in a solution containing 50% deionized formamide, 5×SSC (750 mM sodium chloride, 750 mM sodium citrate), Denhardt's solution, 0.02 M sodium phosphate (pH 6.8), 0.2% sodium dodecyl sulfate (SDS), 100 micrograms of sheared denatured salmon sperm DNA/ml, and 100 micrograms of yeast RNA/ml for 16 h at 58° C. The membranes were washed once in 2×SSC-0.1% SDS for 30 min at room temperature and twice in 0.1×SSC-0.1% SDS for 30 min at 68° C. Membranes were exposed to X-ray film or to a phosphorimaging screen for quantitative analyses with the Fuji BAS 1000 system.

RT-PCR and DNA sequencing. HCV replicons were isolated and cloned from established cell lines by PCR amplification of three fragments spanning the entire NS region including sequences from position 1387 to 7794. DNA synthesis was carried out with SuperScript II RT provided in a cDNA synthesis kit (Gibco-BRL). The DNA oligomers used as primers for the RT reactions mapped to positions 3876 to 3857, 5640 to 5618, and 7794 to 7760, respectively. The reaction mixtures were incubated for 1 h at 45° C. PCR was performed with an Advantage PCR kit (Clontech, Palo Alto, Calif.). One microliter of the cDNA reaction mixture was used for PCR with primers spanning positions 1387 to 3876, 3400 to 5610, and 5538 to 7794. The PCR products were purified by agarose gel electrophoresis and ligated with pGEMT Easy (Promega, Madison, Wis.). Three to six clones of each fragment were sequenced with an ABI automatic DNA sequencer, and a consensus sequence was established with the help of a sequence assembly program (Genetics Computer Group, Madison, Wis.).

In vitro translation. In vitro translation reactions were carried out with micrococcal-nuclease-treated rabbit reticulocyte lysates (Promega) in the presence of [³⁵S]methionine in accordance with the manufacturer's protocol. Translation products were electrophoresed through SDS-10% polyacrylamide gels. The gel was fixed with 30% methanol and 10% glacial acetic acid, treated with NAMP100 Amplify solution (Amersham), dried, and exposed to X-ray film.

### Results

Adaptive mutations in cell lines expressing HCV subgenomes. To conduct our studies, we used a chemically synthesized replica of the subgenome, I377/NS3-3′, described by Lohmann et al. (Science 285: 110-113 (1999)) (FIG. 3). This clone expresses an NS5A protein that is associated with an IFN-alpha-resistant phenotype (Blight, et al. (2000) Science 290:1972-1975). Electroporation of Huh7 cells in the presence of RNA transcribed from linearized I377/NS3-3′ plasmids resulted in the selection of G418-resistant colonies essentially as reported previously (Lohmann, et al. (1999) Science 285:110-113). From four independent experiments we estimated that approximately 1 to 5 out of 10⁶ cells transfected with 10 micrograms of RNA survived the selection process. We expanded 20 clones into cell lines for the analysis of viral RNA. All 20 cell lines contained subgenomic viral RNA of positive as well as negative polarity. RNA expression levels ranged from approximately 200 to 2,000 copies of plus strand RNA per cell, depending on the cell line examined (FIG. 6B, lanes 1 to 3). Moreover, we found that the accumulation of viral RNA was strongly influenced by cell density and, by inference, by the cell cycle. The levels of viral RNA in subconfluent cells were approximately 10-fold higher than those in confluent cells.

We identified consensus mutations in cDNAs obtained from replicons present in the four cell lines examined (Table II). Two cell lines, FCA22 and FCC2, contained an identical mutation changing lysine 821 in NS4B to threonine. Line FCA4 contained only one consensus mutation, a deletion of serine 1176 in NS5A, and FCA1 contained several consensus mutations in NS3 as well as in NS5A. To examine whether the consensus mutations conferred an adapted phenotype, we constructed variants BM4-5, BMB22-2, and BMB2-2, carrying the consensus mutations observed in cell lines FCA4, FCA22, and FCC2, respectively (Table III). Transfection of Huh7 cells with these mutants resulted in the appearance of many colonies, ranging from approximately 250 (BMB22-2) to 3,500 (BM4-5) colonies per mg of RNA (FIG. 6A, Table III). This represented an increase of the transduction efficiency of 3 to 4 orders of magnitude compared to that for the parent I377/NS3-3′ construct (Table III). Viral RNA levels in pools of cells transfected with the adapted mutants were similar to those in the parental cell lines, indicating that the nature of the mutations could influence the level of viral replication.

TABLE II
Summary of nucleotide sequence analysis of cDNAs derived from replicons in
Huh7 cell lines

| Clone | Mutation in^{a}: | | Frequency^{b} | Adapted mutant |
|---|---|---|---|---|
| | cDNA (nt) | Encoded protein^{c} | | |
| FCA1 | G to A (1875) | None (25) NS3 | 6/6 | |
| | A to G (2330) | E to G (177) NS3 | 4/6 | |
| | C to G (5487) | D to E (1229) NS5A | 3/3 | |
| | A to G (5695) | I to V (1299) NS5A | 3/3 | |
| FCA4 | Del. TCA (5327-5329) | Del. S (1176) NS5A | 3/3 | BM4-5 |
| FCA22 | T to C (1777) | None (NA^{e}) EMCV IRES | 3/3 | |
| | A to C (4262) | K to T (821) NS4B | 3/3 | BMB22-2 |
| | C to T (6881) | A to V (1694) NS5B | 2/3 | |
| FCC2 | A to T (4089) | A (763) NS4B | 3/3 | BMB2-2^{d} |
| | A to C (4262) | K to T (821) NS4B | 3/3 | |

^{a}Nucleotide and amino acid positions are according to the sequence of the HCV subgenomic replicon I377/NS3-39 (GenBank accession no. AJ242652). The numbering of the amino acids begins with the artificial methionine of the NS region. nt, nucleotide(s); del., deletion.
^{b}Fraction of PCR-amplified cDNA clones that carry the mutation.
^{c}Order of entries is as follows: mutation (amino acid position) protein designation.
^{d}Contains both mutations in FCC2.
^{e}NA, not applicable.

TABLE III
Summary of RNA transfection experiments

| Construct | Genotype | No. of colonies/mg of RNA |
|---|---|---|
| I₃₇₇/NS3-3″ | lb | 0.1-0.5 |
| BM4-5 | lb | 3,455 |
| BMB2-2 | lb | 644 |
| BM22-2 | lb | 251 |
| HCVlbneo | lb | 235-470 (352) |
| D2Rneo | la | ND^{a} |

^{a}ND, not detected.

Replication of a subgenomic replicon derived from an infectious HCV clone. This study and previous ones relied on an HCV clone that has not yet been tested for infectivity in a chimpanzee. Hence, it is possible that the low transduction efficiency observed with I377/NS3-3′ is due to a defect in the NS protein-coding region that must be corrected to achieve efficient RNA replication in Huh7 cells. In addition, it is not known whether HCV clones encoding NS5A associated with an IFN-alpha-sensitive phenotype can replicate in Huh7 cells. Therefore, we designed two additional constructs using known infectious HCV clones representing HCV subtypes 1a and 1b (HCV1a and 1b). In the HCV1a-based replicon, the 5′ and 3′ untranslated regions as well as the entire NS protein-coding region were derived from infectious HCV clone H77 (Kolykhalov, et al. (1997) Science 277:570-574). In the HCV1b-based replicon, HCV1bneo, the entire NS protein-coding region of I377/NS3-3′, beginning at a BsrG1 restriction site located 225 nucleotides downstream from the 59 end of the NS3 coding sequence, was replaced with the corresponding region of infectious clone HCV-N (FIG. 3) (Beard, et. al (1999) Hepatology 30:316-324). Due to the design of the construct, the NS3 protein encoded by HCV1bneo differed from NS3 of HCV-N at two amino acids (amino acid [aa] 27, Arg in place of Lys; aa 73, Thr in place of Ala).

While transfection of Huh7 cells with HCV1a-based replicon D2Rneo did not yield any G418-resistant colonies, transfection with HCV1bneo resulted in the selection of approximately 350 colonies per microgram of RNA (FIG. 7A; Table III). Hence the transduction efficiency observed with HCV1bneo was similar to those of adapted variants BMB22-2 and BMB2-2 described above and about 700- to 3,500-fold higher than that observed with RNA derived from I377/NS3-3′. An analysis of HCV1bneo RNA present in G418-resistant cells derived from a pool of an estimated 2,000 colonies revealed about 200 copies of RNA per cell, similar to the levels observed in pools from cells transfected with BMB22-2 and BMB2-2 (FIG. 7B). Similar results were obtained with subgenomic replicon HCVNneo, where the entire NS protein-coding region was derived from HCV-N.

These results showed that a subgenomic replicon based on an infectious HCV1b clone can efficiently establish stable replicons in transfected Huh7 cells. On the other hand, the results indicated that an HCV1a-derived clone was defective in this system. In an attempt to explain this difference between the two subtypes, we performed in vitro translation reactions with RNA derived from I377/NS3-3′, HCV1bneo, HCVNneo, and D2Rneo. Unexpectedly, we noticed a significant difference between the translation efficiencies of the NS polypeptides of the two subtypes (FIG. 7C). With the HCV1b-based constructs, translation levels of the NS proteins were similar to those of the neomycin gene, whereas with D2Rneo translation of the viral proteins was significantly reduced. Thus, our results suggested that sequences in the NS region may somehow influence the activity of the encephalomyocarditis virus (EMCV) internal ribosome entry site (IRES). In apparent agreement with such a possibility, a secondary-structure analysis of sequences spanning the IRES sequence and the first 200 nucleotides of NS3 revealed that the HCV1a clone could potentially form a stem-loop structure with the EMCV IRES. Indeed, substitution of the first 227 nucleotides of the NS region in D2Rneo with the corresponding fragment of HCV1b rescued the translation of the NS proteins (FIG. 7C, lane 5). However, recombinant subgenomic replicon D2Ineo did not yield colonies after transfection of Huh7 cells, suggesting that either some other features of the HCV1a isolate interfered with the establishment of stable replicons or the chimeric NS3 protein was not supporting replication in transfected Huh7 cells.

To determine whether adaptive mutations identified with I377/NS3-3′-based replicons could increase the replication levels of HCV-N-based subgenomes, we introduced the adaptive mutation of replicon BM4-5, leading to the deletion of serine 1176 in NS5A, into HCV1bneo. In vitro-transcribed RNA from the resulting replicon, 1bneo/delS, was transfected along with RNA from HCV1bneo, I377/NS3-3′, and BM4-5 into Huh7 cells. RNA transcribed from I377/NS3-3′X, carrying a frameshift mutation in the NS5A coding sequence, served as a negative control. To determine the effect of the mutation in 1bneo/delS on viral replication, we determined the levels of viral RNA 24, 48, and 72 h after transfection (FIG. 8A). This transient assay is based on the previously published observation that adapted replicons can amplify plus strand RNA more efficiently after transfection than the wild type (Blight, et al. (2000) Science 290: 1972-1975; Krieger et. al (2001) J. Virol. 75: 4614-4624). While the BM4-5 replicon exhibited only moderately enhanced RNA levels compared with the wild-type levels, 1bneo/delS showed a much more dramatic increase compared with HCV1bneo (FIG. 8). Hence, these results indicated that adaptation of HCV-N-based replicons can occur in a fashion similar to that observed with I377/NS3-3′.

IFN response in Huh 7 cells. A major question in HCV biology is whether IFN-alpha-based antiviral therapy leads to the selection of IFN-alpha-resistant viral mutants. To address this question, we investigated whether the replication of HCV subgenomes is affected by IFN-alpha. Initial experiments with cell line FCA1 revealed that IFN-alpha reduced the levels of HCV RNA in Huh7 cells in a dose-dependent manner (FIG. 9). Inhibition was observed with IFN-a doses as low as 1 IU/ml, and 10- to 20-fold inhibition was obtained with 25 IU/ml. Of particular interest was the question of whether NS5A had any influence on the IFN response, as has been suggested previously. Because the I377/NS3-3′-based replicon encodes an NS5A protein that is expected to confer IFN resistance, in contrast to the HCV-N-based genomes, we directly compared pools of cells transfected with each replicon for their sensitivities to the antiviral action of the cytokine. Our results showed that both replicons responded in an equal fashion to IFN-alpha, suggesting that under our selected conditions IFN sensitivity is not influenced by variations in NS5A.

Cells supporting HCV replication are thought to harbor plus strand RNA base paired with minus strand RNA in replication complexes as well as excess plus strand RNA derived from the RNA amplification reaction. To examine whether IFN-alpha treatment affected only free plus strand RNA or replication complexes or both, we measured the levels of plus and minus strand RNA in cells treated with 100 IU of IFN-alpha/ml. Our results showed that both RNA strands were reduced with a half-life of about 12 h (FIG. 10). However, the decline of minus strand RNA appeared to be delayed by approximately 12 h, which could reflect differences in the mechanism of RNA degradation between free and complexed RNA.

To examine whether IFN-alpha treatment permitted the selection of IFN-resistant mutants, we maintained cell line FCA1 for over 100 days in the presence of G418 and increasing concentrations of IFN-alpha to a final concentration of 1,000 IU/ml (FIG. 11). Although we noticed that these conditions reduced the growth rate of the cells, we did not observe massive cell death, which occurs during the selection of G418-resistant colonies with subgenomic replicons. Consistent with this observation, viral RNA continued to replicate in IFN-alpha-treated cultures, albeit at approximately 10-fold-reduced levels compared to those for untreated cells. We examined whether IFN-alpha-resistant replicons were present in the FCA1 cells after treatment with increasing doses of the cytokine up to 1,000 IU/ml for 4 months. These cells were incubated for 6 days without the cytokine followed by an incubation with 1 to 100 IU of IFN-alpha/ml for 72 h before viral RNA levels were determined. The results showed that IFN-alpha-treated cells (FCA1/IFN) increased their viral RNA levels to 20% of the RNA level present in the parental FCA1 cell line when IFN-alpha was removed from the culture medium. Further-more, the FCA1/IFN cells exhibited a slightly attenuated dose-response pattern compared with FCA cells (FIG. 11). Whether this difference reflects changes in the response of the cells to IFN-alpha or alterations in the virus is not yet known. Based on the observation that the two cell lines did not differ in terms of the maximal reduction of viral replication (100 IU/ml), we favor the former over the latter possibility.

### Discussion

In this report we demonstrated that a subgenomic replicon based on an infectious HCV1b isolate can replicate in tissue culture cells with an approximately 1,000-fold-higher colony formation efficiency than the previously reported I377/NS3-3′ replicon (Table III). We found that the I377/NS3-3′ replicon undergoes adaptation as a consequence of replication in Huh7 cells.

Moreover, the transfer of adapted mutations can also enhance the RNA replication of related replicons, as shown with the 1bneo/delS replicon (FIG. 8). Whether adaptive mutations occur in cells transfected with wild-type HCV-N-based replicons has not yet been determined. However the results obtained with 1bneo/delS provide support for this possibility.

Our observations have opened the possibility of analyzing HCV isolates from different patient sera for their biological properties in transfected cells. For example, clones from patients taken before and after therapy with IFN and ribavirin can now be isolated and tested for their biological behavior including drug resistance in tissue culture cells. Although the correlation between the expression of the NS proteins from subgenomic RNA templates in vitro and viral replication in cells is incomplete, it could be used as one predictor for the ability of a particular construct to replicate in Huh7 cells (FIG. 7C).

Our results showed that subgenomic replicons based on the I377/NS3-3′ construct are sensitive to the antiviral program induced by IFN-alpha in Huh7 cells (FIGS. 9 to 11). IFN reduced genomic (plus strand) RNA levels with a half-life of approximately 12 h. Interestingly, inhibition of minus strands was delayed by about 12 h, which could suggest that replication complexes are more resistant to degradation by the cellular antiviral response than free plus strand RNA.

Our results showed that I377/NS3-3 and HCV-N-based replicon HCV1bneo behaved almost identically in terms of their response to IFN-alpha (FIG. 9). These observations are surprising considering that I377/NS3-3, in contrast to HCV1bneo, encodes a form of NS5A believed to confer IFN-alpha resistance. It should be noted that the mutations in NS5A identified in cell lines FCA1 and FCA4 (Table 11) are outside of the IFN sensitivity-determining region of NS5A (Blight, et al. (2000) Science 290:1972-1975). However, it is known that the IFN response is multifactorial, and perhaps as a consequence there is, so far, no known virus that has adopted an IFN-resistant phenotype. Our results obtained from the long-term incubation of FCA1 cells with IFN-alpha did not reveal any evidence for a selection of such variants (FIG. 6). Our observation that IFN-treated cells were able to maintain low levels of subgenomic replicons is in good agreement with models predicting the long-term suppression of the IFN response after a relatively short activation period. Nevertheless, our observations should not be interpreted to mean that IFN-alpha-resistant HCV variants cannot be identified in Huh7 cells. For example, their isolation might depend on alternative selection strategies, i.e., the incubation of cell lines such as FCA1 with very high initial doses of IFN-alpha.

### EXAMPLE 3

### Screening Assays for Anti-HCV Therapeutics Using HCV-Replicating Cell Clones of the Present Invention

In the identified efficiently growing “adapted” Huh-7 cell clones containing replicating recombinant HCV genomes, conditions are optimized for cell growth and HCV RNA replication. The timing of exposure to test compounds is determined based on the kinetics of HCV RNA accumulation and/or decline in the absence of the selective agent, and the effects of various known inhibitors of RNA synthesis. A cell-based assay for HCV genome replication is developed based on all these data. In this assay, candidate compounds are tested for inhibition of HCV RNA replication that is selective with respect to inhibition of cell growth. In particular, a reduction of plus or minus strand HCV RNA, or shifting of growth rate curves to a lower growth rate in the presence of a test compound, indicates that the compound is a potential lead for developing an anti-HCV therapeutic.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All values given in the specification are approximate, and are provided for illustration and not by way of limitation.

What is claimed is:

1. An isolated nucleic acid molecule encoding a recombinant HCV genome, comprising the nucleotide sequence of FCA1 (SEQ ID NO: 15).

2. A stable cell line transfected with the isolated nucleic acid molecule of claim 1, wherein said cell line is capable of supporting efficient replication of said isolated nucleic acid.

3. The cell line of claim 2, wherein said cell line is selected from the group consisting of human hepatoma cell line Huh-7, human hepatoma cell line HepG2, hepatoma cell line PH5CH, T. belangeri liver cell line MBTL, human diploid fibroblast cell line VERO, secondary monkey kidney cell line CV-1, T cell line MT-2, T cell line HPBMa10-2, T cell line MOLT-4, and B cell line Daudi.

4. The cell line of claim 3, wherein said cell line is derived from human hepatoma cell line Huh-7.

5. The cell line of claim 4 designated FCA1 and having ATCC Accession No. ______.

6. An isolated nucleic acid molecule encoding a recombinant HCV genome, wherein said nucleic acid molecule comprises at least one consensus mutation of cell line FCA1.

7. A stable cell line transfected with the isolated nucleic acid molecule of claim 6, wherein said cell line is capable of supporting efficient replication of said isolated nucleic acid.

8. The isolated nucleic acid molecule of claim 6, wherein said nucleic acid molecule comprises a G to A mutation at position 1875.

9. The isolated nucleic acid molecule of claim 6, wherein said nucleic acid molecule comprises an A to G mutation at position 2330.

10. The isolated nucleic acid molecule of claim 6, wherein said nucleic acid molecule comprises a C to G mutation at position 5487.

11. The isolated nucleic acid molecule of claim 6, wherein said nucleic acid molecule comprises an A to G mutation at position 5695.

12. An isolated nucleic acid molecule encoding a recombinant HCV genome, comprising the nucleotide sequence of FCA4 (SEQ ID NO: 16).

13. A stable cell line transfected with the isolated nucleic acid molecule of claim 12, wherein said cell line is capable of supporting efficient replication of said isolated nucleic acid.

14. The cell line of claim 13, wherein said cell line is selected from the group consisting of human hepatoma cell line Huh-7, human hepatoma cell line HepG2, hepatoma cell line PH5CH, T. belangeri liver cell line MBTL, human diploid fibroblast cell line VERO, secondary monkey kidney cell line CV-1, T cell line MT-2, T cell line HPBMa10-2, T cell line MOLT-4, and B cell line Daudi.

15. The cell line of claim 14, wherein said cell line is derived from human hepatoma cell line Huh-7.

16. The cell line of claim 15 designated FCA4 and having ATCC Accession No. ______.

17. An isolated nucleic acid molecule encoding a recombinant HCV genome, wherein said nucleic acid molecule comprises at least one consensus mutation of cell line FCA4.

18. The isolated nucleic acid molecule of claim 17, wherein said nucleic acid molecule comprises a deletion of nucleotides 5327-5329.

19. A stable cell line transfected with the isolated nucleic acid molecule of claim 18, wherein said cell line is capable of supporting efficient replication of said isolated nucleic acid.

20. The cell line of claim 19, wherein said cell line is selected from the group consisting of human hepatoma cell line Huh-7, human hepatoma cell line HepG2, hepatoma cell line PH5CH, T. belangeri liver cell line MBTL, human diploid fibroblast cell line VERO, secondary monkey kidney cell line CV-1, T cell line MT-2, T cell line HPBMa10-2, T cell line MOLT-4, and B cell line Daudi.

21. The cell line of claim 20, wherein said cell line is derived from human hepatoma cell line Huh-7.

22. An isolated nucleic acid molecule encoding a recombinant HCV genome, comprising the nucleotide sequence of FCA22 (SEQ ID NO: 17).

23. A stable cell line transfected with the isolated nucleic acid molecule of claim 22, wherein said cell line is capable of supporting efficient replication of said isolated nucleic acid.

24. The cell line of claim 23, wherein said cell line is selected from the group consisting of human hepatoma cell line Huh-7, human hepatoma cell line HepG2, hepatoma cell line PH5CH, T. belangeri liver cell line MBTL, human diploid fibroblast cell line VERO, secondary monkey kidney cell line CV-1, T cell line MT-2, T cell line HPBMa10-2, T cell line MOLT-4, and B cell line Daudi.

25. The cell line of claim 24, wherein said cell line is derived from human hepatoma cell line Huh-7.

26. The cell line of claim 26 designated FCA22 and having ATCC Accession No. ______.

27. An isolated nucleic acid molecule encoding a recombinant HCV genome, wherein said nucleic acid molecule comprises at least one consensus mutation of cell line FCA22.

28. A stable cell line transfected with the isolated nucleic acid molecule of claim 27, wherein said cell line is capable of supporting efficient replication of said isolated nucleic acid.

29. The isolated nucleic acid molecule of claim 29, wherein said nucleic acid molecule comprises a T to C mutation at position 1777.

30. The isolated nucleic acid molecule of claim 29, wherein said nucleic acid molecule comprises a A to C mutation at position 4262.

31. The isolated nucleic acid molecule of claim 29, wherein said nucleic acid molecule comprises a C to T mutation at position 6881.


SEQUENCE LISTING (17 sequences)

SEQ ID NO: 1 - DNA, 7992 bp, Artificial Sequence
  HCV1b-based chimeric replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 2 - DNA, 7992 bp, Artificial Sequence
  HCV2 subgenomic HCV replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctacctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatgtgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcttaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 3 - DNA, 7995 bp, Artificial Sequence
  HCVR8 subgenomic HCV replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaatatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 4 - DNA, 7992 bp, Artificial Sequence
  HCVR9 subgenomic HCV replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaggtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaggccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggctaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaaaggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 5 - DNA, 7992 bp, Artificial Sequence
  HCVR22 subgenomic HCV replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaatcacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcgaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 6 - DNA, 7992 bp, Artificial Sequence
  HCVR24 subgenomic HCV replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 7 - DNA, 22 bp, Artificial Sequence
  RT-PCR primer for NS5B region between nt 7435 and 7750
gccctagattgtcagatctacg

SEQ ID NO: 8 - DNA, 22 bp, Artificial Sequence
  RT-PCR primer for NS5B region between nt 7435 and 7750
ataaatccaactgggacgcagc

SEQ ID NO: 9 - DNA, 21 bp, Artificial Sequence
  RT-PCR primer for NS5B region between 7360 and 7800
ccttgtgggcaaggatgatcc

SEQ ID NO: 10 - DNA, 23 bp, Artificial Sequence
  RT-PCR primer for NS5B region between nt 7360 and 7800
gacaggctgtgatatatgtctcc

SEQ ID NO: 11 - DNA, 22 bp, Artificial Sequence
  RT-PCR primer for neo region between nt 650 and 1110
gttctttttgtcaagaccgacc

SEQ ID NO: 12 - DNA, 21 bp, Artificial Sequence
  RT-PCR primer for neo region between nt 650 and 1110
ccaccatgatattcggcaagc

SEQ ID NO: 13 - DNA, 18 bp, Artificial Sequence
  RT primer for HCVR replicons
ctcgtatgttgtgtggaa

SEQ ID NO: 14 - DNA, 19 bp, Artificial Sequence
  RT primer for HCVR replicons
gtcgctctcgaggcacata

SEQ ID NO: 15 - DNA, 7992 bp, Artificial Sequence
  FCA1 Replicon Sequence
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgagacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggagtctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccggtaccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 16 - DNA, 7989 bp, Artificial Sequence
  FCA4 Replicon Sequence
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact

SEQ ID NO: 17 - DNA, 7992 bp, Artificial Sequence
  FCA22 Replicon Sequence
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggtttccctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgttgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtact